(19) United States
(12) Patent Application Publication
(10) Pub. No.: US 2026/0007693 A1
(43) Pub. Date: Jan. 8, 2026

(54) METHODS AND APPARATUSES FOR DELIVERING GASEOUS NITRIC OXIDE TREATMENTS

(71) Applicant: BEYOND AIR, INC., Garden City, NY (US)

(72) Inventors: Frederick John MONTGOMERY, Sun Prairie, WI (US); Sunil J. PANCHAL, Garden City, NY (US)

(21) Appl. No.: 19/053,541

(22) Filed: Feb. 14, 2025

Related U.S. Application Data
(63) Continuation of application No. PCT/US23/30630, filed on Aug. 18, 2023.
(60) Provisional application No. 63/399,183, filed on Aug. 18, 2022.

(51) Int. Cl. A61K 33/00; A61P 35/00

(52) U.S. Cl. CPC A61K 33/00; A61P 35/00

(57) ABSTRACT

The invention relates to medical gas delivery devices for delivering a medical gas to a tumor mass while minimizing exposure to spent gas or excess gas, wherein the devices comprise a support body coupled with an elongated shaft, wherein the elongated shaft comprises a distal tip structure at its distal end and a selectively permeable diffusion membrane that encloses the distal end of the elongate shaft, and methods for their use. The invention further relates to systems comprising such devices for delivering therapeutic gNO to a tumor mass and kits for use with such systems.

### RELATED APPLICATIONS

This application is a continuation of International Application No. PCT/US2023/030630, which designated the United States and was filed on Aug. 18, 2023, which claims the benefit of priority of U.S. Provisional Application No. 63/399,183, filed on Aug. 18, 2022. The contents of the above applications are incorporated by reference as if fully set forth herein in its entirety.

### FIELD OF THE APPLICATION

The invention generally relates to devices, kits, and methods for delivering therapeutic gaseous nitric oxide (gNO) to a tumor mass.

### BACKGROUND

Cancer is a disease characterized by abnormal cellular proliferation, with the ability of the abnormal cells to invade locally and to spread to other parts of the body. This ability to invade and spread is a salient characteristic of cancer: the emergence of an invasive phenotype in an abnormal cellular proliferation identifies the cell growth as malignant as opposed to benign. Hematological malignancies invade the blood, bone marrow, and lymphatic system. Solid cancers spread by invading local tissues and by metastatic spread. While certain benign tumors can spread locally and invade or displace normal tissues, the potential for metastatic spread is generally a hallmark of a malignant tumor or cancer.

Early detection techniques can be employed to identify and treat malignancies before the dissemination of metastasis. Once a tumor has been detected, therapeutic goals include extirpation or shrinkage of lesions, prevention of metastasis, and treatment of established metastasis. However, about 90% of the mortality from solid cancers is due to metastatic disease itself or due to the complications of treating the metastatic disease systemically. While local or locoregional treatment of the primary tumor is a mainstay of therapy, it does not address the more deadly phenomenon of metastasis. Surgical techniques or locally-directed radiation can only address the primary tumor and its extension into local tissues and into the regional lymphatic system. Tumors that have spread further are deemed metastatic and require systemic therapies.

Chemotherapy and immunotherapy permit treatment of the disseminated spread of a malignancy. These treatments can be used as adjuvants at the same time as or even prior to local control measures (surgery and/or radiation), in an effort to curtail or minimize subsequent metastasis. These treatments can also be employed following surgery or radiation if there is evidence of distal spread. These modalities impose significant burdens on the patient, however. Chemotherapeutic agents are typically cytotoxic, often causing severe and unacceptable side-effects because they kill normal cells as well as malignant ones. Systemic symptoms of toxicity (nausea, vomiting, anorexia, hair loss) and suppression of bone marrow and lymphoid tissue are common with chemotherapies. Immunotherapeutic agents work by stimulating the immune system to attack the disseminated cancer cells and generally have less intense side effects than chemotherapy, with symptoms such as fatigue, fever and chills, weakness, joint pain, headaches, breathing issues and the like, that are caused by the upregulation of the immune system and significant inflammatory reactions that these treatments produce. Moreover, both chemotherapy and immunotherapy may only offer partial treatment for the metastatic disease, instead of a cure. The limitations of chemotherapy are recognized: it is non-specific, acting on all cells that are replicating and not just on the tumor cells; and it does not destroy those cancer cells that are in a resting state. Immunotherapy is a promising alternative, but limited due to its side effects and lack of direct cytotoxic effect on the metastatic foci.

As an alternative approach to conventional anti-metastatic treatment, it has been recognized that the patient's own immune system can be harnessed to attack distant tumor cells by the release of tumor-associated antigens from the primary malignancy. These tumor-associated antigens released locally can activate the immune system to recognize other incidences of these tumor-associated antigens as might be found on metastatic tumor cells. An immune-stimulating technique observed to be effective in combatting metastatic spread is the ablation of the local tumor in a way that releases tumor antigens to stimulate/trigger the immune system response. The immune system can operate systemically, attacking the sites of metastatic spread. The ability of a treatment directed to a local malignancy to trigger an immune response that affects a distant metastasis is called the abscopal effect. Ablation of a malignant tumor mass, whether carried out surgically or through less invasive interventions, can not only remove or curtail tumor growth at the primary site but can cause sufficient tumor-specific immunostimulation that distant disease is treated as well. Since the resection or ablation of the local tumor may not achieved completely, with residual macroscopic or microscopic disease, the tumor-specific immunostimulation triggered by the release of tumor-associated antigens from the primary malignancy can combat residual disease at the primary site as well as attacking distant metastases.

Nitric oxide in its gaseous form (gNO) is toxic to living cells when delivered in high concentration, leading to its consideration as a potential anti-tumor therapy because of its potential to kill malignant cells as well as non-malignant ones. Nitric oxide is understood to induce cell death by activating the ASK1/JNK1 (apoptosis signal-regulating kinase 1/c-Jun N-terminal kinase 1) pathway, which leads to intrinsic cell apoptosis. NO thus can have an ablative effect on local tumor cells, with the potential for triggering the abscopal effect. The use of NO as an anti-cancer therapy has been limited to date because of its overall cytotoxicity: NO is toxic to both normal and malignant cells when administered in sufficient concentrations to have an anti-tumor effect. A significant limitation in its use, therefore, has been an accurate delivery method that directs NO to impact just the tumor cells, while permitting the retrieval of this substance before it damages the healthy cells in the vicinity. It is understood that a high concentration of gaseous NO (gNO) induces cytotoxic effects when effectively delivered to tumor cells, but it is recognized that such a high concentration will also damage the local tissues.

There remains a need in the art, therefore, for an appropriately dimensioned delivery device that permits the measured and targeted delivery of high concentrations and appropriate volumes of gNO to the site of a local tumor. There remains a concomitant need for a NO scavenging apparatus that removes the NO from the local site before it exerts its cytotoxic effects on adjacent healthy tissues. Advantageously, such a device would be suitable for use with multiple extirpative or ablative technologies, including open surgical resection and the multitude of non-invasive treatment modalities.

### BRIEF DESCRIPTION OF FIGURES

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to provide drawings to scale, or to show structural details of the invention in more detail than is necessary for a fundamental understanding of the invention, the description taken with the drawings making apparent to those skilled in the art how the several forms of the invention may be embodied in practice.

FIGS. 1A and 1B are schematic diagrams of a gNO treatment system in accordance with the systems and methods disclosed herein.

FIGS. 2A and 2B are schematic diagrams of a gNO treatment system in accordance with the systems and methods disclosed herein.

FIG. 3 is a schematic diagram of a delivery device suitable for use with the systems and methods disclosed herein.

FIGS. 4A, 4B, 4C, and 4D are schematic diagrams of a distal portion of a delivery device suitable for use with the systems and methods disclosed herein.

FIG. 5A and cross-section FIG. 5B depict an embodiment of a delivery device suitable for use with the systems and methods disclosed herein. FIG. 5C provides a close-up view of a section of the delivery device depicted in FIG. 5B.

FIG. 6A and cross-section FIG. 6B depict another embodiment of a delivery device suitable for use with the systems and methods disclosed herein.

### SUMMARY

Disclosed herein, in embodiments, are systems for delivering therapeutic gNO to a tumor mass while minimizing exposure to spent gas or excess gas, comprising a source of therapeutic gNO, a gas collector, and a gas delivery device comprising a support body coupled with an elongate shaft, wherein the elongate shaft has a proximal end and a distal end, wherein the distal end of the elongate shaft is positionable in a treatment region in therapeutic proximity to the tumor mass; wherein the elongate shaft is in fluid communication with the source of therapeutic gNO, and wherein the elongate shaft is adapted for delivering the therapeutic gNO through the distal end of the elongate shaft into the treatment region; and wherein the elongate shaft is in fluid communication with the gas collector, and wherein the elongate shaft is adapted for scavenging gas through the distal end of the elongate shaft, wherein the scavenged gas is spent gas or excess gas; wherein the therapeutic gNO flows distally from the source of therapeutic gNO into the distal end of the elongate shaft and therefrom into the treatment region, thereby accessing the tumor mass, and wherein any spent or excess gas present in the delivery device, for example any gas present in the elongate shaft that does not diffuse across the membrane into the tissue, is scavenged to minimize exposure thereto.

In embodiments, the elongate shaft comprises a single channel, wherein, when gas is delivered to the tumor, the single channel provides a path for the therapeutic gas (e.g., therapeutic gNO) to flow distally from the source of therapeutic gas into the distal end of the elongate shaft and therefrom into the treatment region and, after the gas has been delivered, any spent or excess gas present in the delivery device, for example any gas present in the single channel of the elongate shaft that does not diffuse across the membrane into the tissue, is scavenged.

In embodiments, the elongate shaft comprises two or more channels, wherein at least one channel is an insufflation channel that passes from proximal to distal within the elongate shaft and is adapted for delivering the therapeutic gNO through the distal end of the elongate shaft into the treatment region; and wherein at least one channel is a gas return channel that passes from distal to proximal within the elongate shaft and is adapted for scavenging gas through the distal end of the elongate shaft, wherein the gas is spent gas or excess gas. In embodiments, the elongate shaft comprises an insufflation channel and a gas return channel. The insufflation channel and the gas return channel are further described herein.

In embodiments, the elongate shaft comprises an insufflation channel that passes from proximal to distal within the elongate shaft, wherein the insufflation channel is in fluid communication with the source of therapeutic gNO, and wherein the insufflation channel is dimensionally adapted for delivering the therapeutic gNO through the distal end of the elongate shaft into the treatment region; and wherein the elongate shaft further comprises a gas return channel that passes from distal to proximal within the elongate shaft, wherein the gas return channel is in fluid communication with the gas collector, and wherein the gas return channel is dimensionally adapted for scavenging gas through the distal end of the elongate shaft, wherein the gas is spent gas or excess gas; wherein the therapeutic gNO flows distally from the source of therapeutic gNO through the insufflation channel into the distal end of the elongate shaft and therefrom into the treatment region, thereby accessing the tumor mass, and wherein the gas passes from the treatment region into the distal end of the elongate shaft and therefrom into the gas return channel, to flow proximally into the gas collector, thereby delivering therapeutic gNO to a tumor mass while scavenging spent gas or excess gas to minimize exposure thereto.

In embodiments, (a) the support body comprises an inflow socket adapted for connection with a gas flow inlet assembly, wherein the gas flow inlet assembly is connectable proximally to the source of therapeutic gNO and is connectable distally to the inflow socket, wherein the gas flow inlet assembly defines a gas flow inlet passage within, and wherein the gas flow inlet passage establishes fluid communication between the source of therapeutic gNO and the elongate shaft when the gas flow inlet assembly is connected to the inflow socket; and (b) the support body comprises an outflow socket adapted for connection with a gas return outlet assembly, wherein the gas return outlet assembly is connectable proximally to the gas collector and is connectable distally to the outflow socket, wherein the gas return outlet assembly defines a gas return outlet passage within, and wherein the gas return outlet passage establishes fluid communication between the gas collector and the elongate shaft when the gas flow outlet assembly is connected to the outflow socket. In embodiments, the gas flow inlet assembly is connected to the inflow socket through a first screw connection, and a first O-ring is positioned at the base of the inflow socket, wherein the first O-ring is compressible by the first screw connection. In embodiments, the gas return outlet assembly is connected to the outflow socket through a second screw connection, and a second O-ring is positioned at the base of the outflow socket, and wherein the second O-ring is compressible by the second screw connection.

In embodiments, the support body encloses the proximal end of the elongate shaft. The proximal end of the elongate shaft can comprise a flared head dimensionally adapted for seating in a distal end of the inflow socket. In embodiments, the flared head can be formed from a compressible material. In embodiments, when seated in the distal end of the inflow socket, the elongate shaft extends from its proximal end through the support body to pass through a distal end of the support body and protrude therefrom. In embodiments, the elongate shaft is secured within the distal end of the support body by a cylindrical scaling fastener, and the cylindrical scaling fastener can house a female screw socket; in embodiments, the distal end of the support body is shaped in a male-to-female screw configuration adapted for a male-to-female screw connection with the female screw socket, so that the elongate shaft can be secured within the distal end of the support body by the screw connection. In embodiments, a third O-ring is positioned at the base of the female screw socket, wherein the third O-ring is compressible by the male screw connection. The elongated shaft can be rigid over its entire length, or it can be flexible over at least a portion of its length. The elongate shaft can be dimensionally adapted for positioning through an endoscope to access the treatment region.

In embodiments, the elongate shaft comprises a distal tip structure at its distal end, wherein the distal tip structure comprises a distal end of the elongate shaft and a selectively permeable diffusion membrane that encloses the distal end of the elongate shaft. In embodiments, the distal tip structure further comprises one or more gas access spaces in fluid communication with the distal end of the elongate shaft and the diffusion membrane can enclose the distal end of the elongate shaft and the one or more gas access spaces. In embodiments, the distal end of the elongate shaft is penetrated with one or more openings on its side providing the fluid communication between the distal end of the elongate shaft and the one or more gas access spaces, and the distal end of the elongate shaft can be sealed. In embodiments, the one or more gas access spaces form a single shared gas access space, which is in fluid communication with the elongate shaft. In embodiments, the system further comprises a gas collector in fluid communication with the elongate shaft.

In embodiments, the elongate shaft comprises a distal tip structure at its distal end, wherein the distal tip structure comprises a distal end of the insufflation channel, a distal end of the gas return channel, and a selectively permeable diffusion membrane that encloses the distal end of the insufflation channel and the distal end of the gas return channel. In embodiments, the distal end of the insufflation channel and the distal end of the gas return channel are approximately even with each other; in other embodiments, the distal end of the insufflation channel extends more distally than the distal end of the gas return channel. In embodiments, the distal tip structure further comprises one or more gas access spaces in fluid communication with the distal end of the insufflation channel and the distal end of the gas return channel, and the diffusion membrane can enclose the distal end of the insufflation channel, the distal end of the gas return channel, and the one or more gas access spaces. In embodiments, the distal end of the insufflation channel is penetrated with one or more openings on its side providing the fluid communication between the distal end of the insufflation channel and the one or more gas access spaces, and the distal end of the insufflation channel can be sealed. In embodiments, the one or more gas access spaces form a single shared gas access space, which is in fluid communication with the insufflation channel and the gas return channel. In other embodiments, the one or more gas access spaces comprise an insufflation gas access space in fluid communication with the insufflation channel and a return gas access space in fluid communication with the gas return channel, and wherein the insufflation gas space and the return gas access space are not in fluid communication with each other. In embodiments, the insufflation channel and the gas return channel are arranged parallel to each other within the elongate shaft, and they can be arranged coaxially or eccentrically within the elongate shaft. In embodiments, the insufflation channel is disposed within the gas return channel. In embodiments, the system further comprises a gas collector in fluid communication with the gas return channel.

Also disclosed herein, in embodiments, are kits for use with the foregoing systems, comprising a support body of a gas delivery device, and an elongate shaft of a gas delivery device insertable into the support body to form the gas delivery device, wherein each of the support body and the elongate shaft is provided in the kit as a separate component. In embodiments, at least one of the support body and the elongate shaft is designed for single use. In embodiments, the elongate shaft comprises an inner insufflation member (also referred to herein as a insufflation channel) and an outer gas return sleeve (also referred to herein as a gas return channel), wherein the inner insufflation member is dimensionally adapted for insertion into the outer gas return sleeve, and wherein each of the inner insufflation member and the outer gas return sleeve is provided in the kit as a separate sub-component, assemblable to form the elongate shaft; one or both of these subcomponents can be designed for single use. In embodiments, the kit further comprises a gas flow inlet assembly and a gas return outlet assembly, each insertable into a socket of the support body, and further comprising a cylindrical scaling fastener adapted for attachment to a distal end of the support body, wherein each of the gas flow inlet assembly, the gas return outlet assembly, and the cylindrical sealing fastener is provided separately in the kit as an auxiliary component. In embodiments, at least one of the gas flow inlet assembly, the gas return outlet assembly, and the cylindrical sealing fastener can be designed for single use. The kit can further comprise one or more O-rings dimensionally adapted for insertion in one or more sockets of the support body or for insertion into the cylindrical sealing fastener; one or more of the O-rings can be designed for single use. In embodiments, the kit further comprises a container housing the support body and the elongate shaft as sterile components.

Also disclosed herein, in embodiments, are kits for use with the foregoing systems, comprising a support body of a gas delivery device, and an elongate shaft of a gas delivery device insertable into the support body to form the gas delivery device, wherein each of the support body and the elongate shaft is provided in the kit as a separate component. In embodiments, at least one of the support body and the elongate shaft is designed for single use. In embodiments, the kit further comprises a gas flow inlet assembly and a gas return outlet assembly, each insertable into a socket of the support body, and further comprising a cylindrical scaling fastener adapted for attachment to a distal end of the support body, wherein each of the gas flow inlet assembly, the gas return outlet assembly, and the cylindrical sealing fastener is provided separately in the kit as an auxiliary component. In embodiments, at least one of the gas flow inlet assembly, the gas return outlet assembly, and the cylindrical sealing fastener can be designed for single use. The kit can further comprise one or more O-rings dimensionally adapted for insertion in one or more sockets of the support body or for insertion into the cylindrical sealing fastener; one or more of the O-rings can be designed for single use. In embodiments, the kit further comprises a container housing the support body and the elongate shaft as sterile components.

In additional embodiments, the disclosure includes medical gas delivery devices for delivering therapeutic gaseous nitric oxide (gNO) to a tumor mass while minimizing exposure to spent gas or excess gas, the device comprising a support body coupled with an elongate shaft, wherein the elongate shaft comprises an insufflation channel that passes from proximal to distal within the elongate shaft; wherein the elongate shaft further comprises a gas return channel that passes from distal to proximal within the elongate shaft; wherein the elongate shaft comprises a distal tip structure at its distal end, and wherein the distal tip structure comprises a distal end of the insufflation channel and a distal end of the gas return channel; and wherein a selectively permeable diffusion membrane encloses the distal end of the insufflation channel and the distal end of the gas return channel. In embodiments, the distal end of the insufflation channel in the device and the distal end of the gas return channel are approximately even with each other, while in other embodiments, the distal end of the insufflation channel extends more distally than the distal end of the gas return channel. The distal tip structure of the device can further comprise one or more gas access spaces in fluid communication with the distal end of the insufflation channel and the distal end of the gas return channel. In embodiments, the distal end of the insufflation channel contains one or more openings in its side for gas delivery, and the distal end of the insufflation channel can be closed at the end and rounded or pointed. In embodiments, the one or more gas access spaces of the device form a single shared gas access space, and the single shared gas access space is in fluid communication with the insufflation channel and the gas return channel. The insufflation channel and the gas return channel can be arranged parallel to each other within the elongate shaft; the insufflation channel and the gas return channel can be arranged coaxially within the elongate shaft; the insufflation channel can be disposed within the gas return channel; the insufflation channel and the gas return channel can be arranged eccentrically within the elongate shaft. In embodiments, the support body of the device comprises: (a) an inflow socket adapted for connection with a gas flow inlet assembly, wherein the gas flow inlet assembly defines a gas flow inlet passage within, and wherein the gas flow inlet passage establishes fluid communication between a source of medical gas and the insufflation channel; and (b) an outflow socket adapted for connection with a gas return outlet assembly, wherein the gas return outlet assembly defines a gas return outlet passage, and wherein the gas return outlet passage establishes fluid communication between a gas collector and the gas return channel. In embodiments, the gas flow inlet assembly is connectable proximally to the source of medical gas and is connectable distally to the inflow socket and the gas return outlet assembly is connectable proximally to the gas collector and is connectable distally to the outflow socket. In embodiments, the medical gas is therapeutic gaseous nitric oxide (gNO). In embodiments, the gas flow inlet assembly is connected to the inflow socket through a first screw connection, and a first O-ring can be positioned at the base of the inflow socket, and the first O-ring can be compressible by the first screw connection. In embodiments, the gas return outlet assembly is connected to the outflow socket through a second screw connection, and a second O-ring can be positioned at the base of the outflow socket, and the second O-ring can be compressible by the second screw connection. In embodiments, the support body encloses the proximal end of the elongate shaft of the device. In embodiments, the proximal end of the elongate shaft comprises a flared head dimensionally adapted for seating in a distal end of the inflow socket, and the flared head can be formed from a compressible material. When seated in the distal end of the inflow socket, the elongate shaft extends from its proximal end through the support body to pass through a distal end of the support body and protrudes therefrom. In embodiments, the elongate shaft of the device can be secured within the distal end of the support body by a cylindrical sealing fastener. In embodiments, the cylindrical sealing fastener houses a female screw socket, and the distal end of the support body is shaped in a male screw configuration adapted for a male-to-female screw connection with the female screw socket, wherein the elongate shaft is secured within the distal end of the support body by the male-to-female screw connection. In embodiments, a third O-ring can be positioned at the base of the female screw socket, and the third O-ring can be compressible by the male-to-female screw connection. In embodiments, the elongate shaft is rigid over its entire length; in other embodiments, the elongate shaft is flexible over at least a portion of its length. The elongate shaft can be dimensionally adapted for positioning through an endoscope to access the treatment region. In embodiments, the one or more gas access spaces comprise an insufflation gas access space in fluid communication with the insufflation channel and a return gas access space in fluid communication with the gas return channel, and the insufflation gas space and the return gas access space need not be in fluid communication with each other.

In additional embodiments, the disclosure includes medical gas delivery devices for delivering therapeutic gaseous nitric oxide (gNO) to a tumor mass while minimizing exposure to spent gas or excess gas, the device comprising a support body coupled with an elongate shaft; wherein the elongate shaft comprises a distal tip structure at its distal end, and wherein a selectively permeable diffusion membrane encloses the distal end of the elongate shaft. The distal tip structure of the device can further comprise one or more gas access spaces in fluid communication with the distal end of the elongate shaft. In embodiments, the distal end of the elongate shaft contains one or more openings in its side for gas delivery, and the distal end of the elongate shaft can be closed at the end and rounded or pointed. In embodiments, the one or more gas access spaces of the device form a single shared gas access space, and the single shared gas access space is in fluid communication with the elongate shaft. In embodiments, the support body of the device comprises: (a) an inflow socket adapted for connection with a gas flow inlet assembly, wherein the gas flow inlet assembly defines a gas flow inlet passage within, and wherein the gas flow inlet passage establishes fluid communication between a source of medical gas and the elongate shaft; and (b) an outflow socket adapted for connection with a gas return outlet assembly, wherein the gas return outlet assembly defines a gas return outlet passage, and wherein the gas return outlet passage establishes fluid communication between a gas collector and the elongate shaft. In embodiments, the gas flow inlet assembly is connectable proximally to the source of medical gas and is connectable distally to the inflow socket and the gas return outlet assembly is connectable proximally to the gas collector and is connectable distally to the outflow socket. In embodiments, the medical gas is therapeutic gaseous nitric oxide (gNO). In embodiments, the gas flow inlet assembly is connected to the inflow socket through a first screw connection, and a first O-ring can be positioned at the base of the inflow socket, and the first O-ring can be compressible by the first screw connection. In embodiments, the gas return outlet assembly is connected to the outflow socket through a second screw connection, and a second O-ring can be positioned at the base of the outflow socket, and the second O-ring can be compressible by the second screw connection. In embodiments, the support body encloses the proximal end of the elongate shaft of the device. In embodiments, the proximal end of the elongate shaft comprises a flared head dimensionally adapted for seating in a distal end of the inflow socket, and the flared head can be formed from a compressible material. When seated in the distal end of the inflow socket, the elongate shaft extends from its proximal end through the support body to pass through a distal end of the support body and protrudes therefrom. In embodiments, the elongate shaft of the device can be secured within the distal end of the support body by a cylindrical sealing fastener. In embodiments, the cylindrical sealing fastener houses a female screw socket, and the distal end of the support body is shaped in a male screw configuration adapted for a male-to-female screw connection with the female screw socket, wherein the elongate shaft is secured within the distal end of the support body by the male-to-female screw connection. In embodiments, a third O-ring can be positioned at the base of the female screw socket, and the third O-ring can be compressible by the male-to-female screw connection. In embodiments, the elongate shaft is rigid over its entire length; in other embodiments, the elongate shaft is flexible over at least a portion of its length. The elongate shaft can be dimensionally adapted for positioning through an endoscope to access the treatment region.

Also disclosed herein, in embodiments, are methods of treating a tumor mass with a therapeutically effective amount of therapeutic gNO, comprising: providing the system described above; positioning the gas delivery device so that the distal end of the elongate shaft is within the treatment region; coupling the delivery device to the source of therapeutic gNO and to the gas collector; directing a flow of therapeutic gNO from the source of therapeutic gNO into the delivery device to pass through the distal end of the elongate shaft into the treatment region to reach the tumor mass; continuing the flow for a preselected time period to expose the tumor mass to the therapeutically effective amount; terminating the flow following delivery of the therapeutically effective amount; and scavenging gas from the treatment region. It is understood that, in some embodiments, the step of coupling the delivery device can take place before the step of positioning the delivery device, while in other embodiments, the delivery device is positioned first, followed by the step of coupling it to the source of therapeutic gNO and the gas collector, as would be appreciated by artisans of ordinary skill in the field. In embodiments, the therapeutically effective amount of therapeutic gNO is a high dose of gNO. In embodiments, the step of positioning occurs during an open surgical procedure, while in other embodiments, the step of positioning comprises a substep of inserting the elongate shaft through an endoscope, which can be positioned to access the treatment region prior to performing the substep of inserting the elongate shaft through the endoscope. At least a portion of the endoscope can be flexible and at least a portion of the elongate shaft can be flexible.

In embodiments, the step of scavenging comprises: (a) directing the gas to enter the distal end of the elongate shaft; and (b) directing the gas to flow proximally into the gas collector. In embodiments, the gas is spent gas or excess gas. The step of scavenging can include a substep of exposing the treatment region to a low-pressure environment, wherein the low-pressure environment directs the spent gas to enter the distal end of the elongate shaft and flow proximally into the gas collector. The step of scavenging can take place after the step of directing the flow, or after the step of continuing the flow, or after the step of terminating the flow, or can take place simultaneously with the step of directing the flow or with the step of continuing the flow. In embodiments, the delivery device comprises a diffusion membrane at its tip to facilitate the step of scavenging gas simultaneously with the step of directing the flow or continuing the flow, and the diffusion membrane can enclose the distal end of the elongate shaft. In embodiments, the step of scavenging can be adjusted to remove gas from the treatment region in adequate amount to minimize the impact of the high dose of gNO on the treatment region.

### DETAILED DESCRIPTION

### Section 1: Overview and Definitions

The present invention provides systems and methods for treating tumors or cancers by local administration of a clinically appropriate dose of gNO. As used herein, “treating” or “treatment” refers to any indicia of success in extirpating or decreasing tumor mass, or in improving the clinical symptoms resulting from such tumor mass. Treating can include, for example, reducing or alleviating the severity of one or more symptoms of the disease or condition, or it can include reducing the frequency with which symptoms of a disease, defect, disorder, or adverse condition, and the like, are experienced by a patient. In embodiments, “treat” or “treating” means accomplishing one or more of the following: (a) reducing tumor size; (b) reducing tumor growth rate; or (c) reducing or limiting development and/or spreading of metastases. The inventive systems and methods of treatment involve a local administration of gNO, which involves the delivery of the gNO to a region in relation to the tumor within which the gNO can exert its therapeutic effect. The inventive systems and methods of treatment further involve the local administration of a clinically appropriate dose of gNO, where the gNO itself is clinically suitable, i.e., is of sufficient purity and sterility to be used in a patient (such gNO that is clinically suitable being termed “therapeutic gNO” herein), and where such therapeutic gNO is delivered at a dose that is adequate for producing the desired treatment without untoward reactions or side effects. In embodiments, the dose is a high dose of gNO, as described below.

Cell growth in general can occur in malignant or benign contexts. The term is typically associated with growth in cell numbers, which occurs by means of cell reproduction (i.e., proliferation) when the rate of the latter is greater than the rate of cell death (e.g., by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered. “Tumor growth” or “tumor mass growth,” as used herein, unless otherwise indicated, is used in the way that it is commonly used in oncology, where the term is principally associated with an increased mass or volume of the tumor or tumor mass, primarily as a result of tumor cell growth. Cancerous (malignant) tumors are characterized by malignant cell growth, which refers to cell growth that is independent of normal regulatory mechanisms (e.g., loss of contact inhibition). This includes the abnormal growth of: (1) tumor cells (tumors) that proliferate by expressing a mutated tyrosine kinase or over-expression of a receptor tyrosine kinase; (2) benign and malignant cells of other proliferative diseases in which aberrant tyrosine kinase activation occurs; (3) any tumors that proliferate by receptor tyrosine kinases; (4) any tumors that proliferate by aberrant serine/threonine kinase activation; and (5) benign and malignant cells of other proliferative diseases in which aberrant serine/threonine kinase activation occurs. Treating a malignant tumor or a cancer can involve an interference with or an impairment of malignant cell growth.

The phrase “a method of treating” or its equivalent, when applied to, for example, cancer, refers to a procedure or course of action that is designed to reduce or eliminate the number of cancer cells in an animal, or to alleviate the symptoms of a cancer. Embarking on a “method of treating” a malignant or benign tumor does not necessarily mean that the abnormal cells will, in fact, be eliminated, that the number of abnormal cells will, in fact, be reduced, or that the consequent symptoms of the malignant or benign tumor will, in fact, be alleviated. Often, a method of treating cancer will be performed even with a low likelihood of success, but which, given the medical history and estimated survival expectancy of a patient, is nevertheless deemed an overall beneficial course of action. As used herein, “preventing” refers to the prevention of a clinical disease or condition, e.g., clinically apparent tumor formation, in the patient. For example, if an individual at risk of developing a tumor mass, or at risk of converting a benign tumor into a malignant version thereof, is treated with the methods of the present invention and does not later form the tumor mass or experience the conversion of the benign tumor to a malignant form, then the disease has been prevented in that individual. As an illustrative example, “preventing” includes inhibiting the growth, spread, and development of cancerous cell phenotypes and growths.

The present invention pertains to delivering the gNO directly into a local primary tumor or a locus of metastatic tumor cells (e.g., a “tumor mass”), or contacting the tumor mass, or delivering gNO into sufficient proximity to the tumor mass that the gNO can exert a therapeutic effect on the tumor mass. The therapeutic effect is any effect that accomplishes a treatment of the local tumor or distant site of tumor metastasis. The amount of the gNO that produces the therapeutic effect is the therapeutic amount of gNO. In more detail, as used herein, the term “therapeutically effective amount” is the amount of gNO sufficient to provide a therapeutic effect to the individual in need thereof (such human or nonhuman individual subject also being termed a patient). For the purposes of these systems and methods, the therapeutic effect achieved by the administration of gNO can be a reduction in the number of viable tumor cells in the patient; this therapeutic effect is termed a tumoricidal effect. A therapeutically effective amount of gNO delivered over a designated time period can reduce the number of viable tumor cells in the tumor mass, whether local or distant. The tumoricidal effect can be observed locally by the direct tumoricidal effect of the gNO, or it can be observed at a distal site, with the decrease in the mass of one or more distant metastases. In embodiments, the tumoricidal effect can be an abscopal effect, in which the local damage to the tumor activates the immune system sufficiently that it acts to decrease the number of viable tumor cells in the distant metastases. This can be experienced by the patient as a regression or obliteration of established metastatic disease. If the distant metastases are in a subclinical or undetectable state, for example as micrometastases, the tumoricidal effect of the gNO on these distant but subclinical metastases can prevent them from becoming established as tumor masses in the distant sites. In this way, the gNO can be viewed as “preventing” the distant metastases, because the subclinical tumor dissemination is sufficiently eradicated that it does not become clinically apparent.

The region in relation to the tumor mass within which the gNO can exert its therapeutic effect is termed the “tumor target,” and the degree of closeness to the tumor mass to exert a therapeutic effect is termed “therapeutic proximity” to the tumor mass. The tumor target is the region in sufficient therapeutic proximity to the tumor mass that gNO delivered into this region can exert a therapeutic effect on the tumor. The tumor target can include the tumor mass itself, with the gNO being directed at the tumor mass by direct contact, including contacting the surface of the tumor mass, penetrating the substance of the tumor mass, or some combination thereof. Therapeutic proximity thus can include close proximity to the tumor that is external to the tumor with or without actual contact to the tumor surface, and further and preferably can include penetration of the substance of the tumor mass. The gNO can also be directed into a region surrounding the tumor mass, which can be relatively unoccupied or which can contain normal tissues or structures, provided that the delivery of the gNO into this region is adequate to produce the intended therapeutic effect. For the purposes of this invention, the intended therapeutic effect is a cytotoxic effect, with sufficient gNO reaching the tumor mass to damage and kill tumor cells.

While the systems and methods disclosed herein can be advantageously employed for the treatment of malignant tumors (i.e., cancers, which are tumors characterized by uncontrolled growth of abnormal cells with the potential for invading nearby tissues and spreading to distant sites in the body), it is envisioned that these systems and methods can also be employed to treat benign (i.e., non-cancerous) tumors whose local growth impinges upon local tissues, especially in sites where access to the lesion is restricted. As an example, the benign tumors associated with neurofibromatosis can develop at any site in the body, including areas that are relatively inaccessible to complete surgical resection. They can continue growing after partial excision or ablation, encroaching on normal structures and causing pain, disfigurement, local hemorrhage, and neurological deficit. Moreover, these tumors, while benign, can undergo malignant degeneration. For benign tumors having locally aggressive behavior or locally damaging effects, or for such tumors situated in relatively inaccessible anatomic locations, the present invention can offer a relatively less invasive treatment option.

Whether applied to malignant or benign targets, the systems and methods disclosed herein use a delivery device specifically engineered both to effect the delivery of the gNO to the targeted tumor mass and to remove the gNO from the delivery site expeditiously and efficiently, in order to minimize collateral cytotoxicity. The gNO delivery device as disclosed herein is suitable for use with a variety of gNO delivery systems that administer the gNO to the tumor mass in a therapeutically effective amount. Advantageously, the systems and methods disclosed herein can remove delivered gas that does not diffuse across the diffusion membrane or reach the tumor target or the surrounding tissues, as well as gas that reaches the tumor target region but that is not needed to deliver the therapeutically effective dose to the tumor target (being termed “excess gas” herein). The removal of excess gas can take place simultaneously with its delivery, near-simultaneously, or sequentially, with the removal following the delivery at specific intervals. In this way, the scavenging of excess gas can balance the delivery amount so that the appropriate dose of gNO is directed at the tumor target, thereby avoiding overdosing or damaging the surrounding tissues, preventing the buildup of byproducts and exposure of tissues thereto, and relieving excessive gas pressures that can develop in or around the structures (including the tumor target) into which the delivery device has been introduced. Advantageously, the systems and methods disclosed herein not only deliver the therapeutically effective amount to the tumor target region, but also retrieve or scavenge any gas directed towards the tumor target that does not enter the tumor target region, and any extravasated gas that has entered the normal tissues surrounding the tumor target (all of such gas being individually and collectively termed “spent gas”), thereby removing these gases from the tumor target region and its surroundings. By removing excess and spent gas, the invention imparts an additional therapeutic benefit of minimizing the cytotoxic effect of the gNO on the healthy tissues in proximity to the delivery area; this additional therapeutic benefit is termed a tissue-protective effect. Furthermore, the systems and methods disclosed herein can remove any gaseous byproducts of excess gas or spent gas. As used herein, the term “byproducts” associated with gNO administration refer to any gaseous secondary products that are derived from the NO molecule (for example by oxidation) or that integrate the NO molecule: for example, NOx gas molecules are considered gNO byproducts, where “x” can be 1 or 2; other gNO byproducts can be formed following the contact of the gNO with the physiological environment.

To improve clarity, the following definitions also apply to terms used in this disclosure.

As used herein, “a” or “an” may mean one or more than one of an item.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like, for medical and/or laboratory research purposes. Preferably, the subject is a human patient. More preferably, the subject is a human patient that has a malignant or locally aggressive benign tumor.

The term “about” as used herein when referring to a measurable value such as an amount of weight, time, dose, etc. is meant to encompass variations of +/−20% or +/−10% from the specified amount, as such variations are appropriate to perform the disclosed method. In embodiments, the term “about” is meant to encompass variations of +/−5%. In embodiments, the term “about” is meant to encompass variations of +/−1%. In embodiments, the term “about” is meant to encompass variations of +/−0.1%.

Various exemplary compositions and methods are described in order to detail various embodiments. It will be obvious to one skilled in the art that practicing the various embodiments does not require the employment of all or even some of the details outlined herein, but rather that concentrations, times and other details may be modified through routine experimentation. In some cases, well-known methods or components have not been included in the description.

### Section 2: Exemplary Delivery Devices

The delivery device for directing a therapeutically effective amount of gNO to the tumor target is a component of a nitric oxide treatment system, wherein the delivery device is responsible for directing a clinically appropriate gNO inflow into the tumor target and for retrieving gNO from the tumor target area. In embodiments, tumor treatment using these systems and methods, a high dose of gNO can be employed. High dose gNO can include the delivery of gNO in a gas at a concentration of between about 1,000 and 1,000,000 ppm, between 5,000 and 750,000 ppm, between 10,000 and 500,000 ppm, between 15,000 and 250,000 ppm, between 20,000 and 100,000 ppm. In preferred embodiments, the delivery device is compatible with the delivery of high dose gNO. Advantageously, the delivery device also allows (1) the retrieval, removal, or recovery of excess gas following its delivery but before its access to the tumor target or the normal tissues in proximity thereto; and/or (2) the retrieval or removal of spent gas from the vicinity of the tumor target. As used herein, the term “scavenging” can include the retrieval, removal, or recovery of spent gas or excess gas, including without limitation: (1) unused gNO remaining within the distal portion of the delivery device without entering the vicinity of the tumor target, (2) any residual gNO or the byproducts thereof in the treatment area, and/or (3) extra or extravasated gNO that has exited the distal portion of the delivery device and has entered the vicinity of the tumor target or has entered normal tissues in proximity thereto.

A system for gNO treatment thus has a source that provides an inflow of gNO and a gas collector, both in fluid communication with the delivery device itself that is intended for accessing the tumor target, delivering the therapeutically effective amount of gNO to the tumor target, and scavenging gNO and byproducts. The source of gNO can be any vessel, conduit, or system containing or delivering gNO at the requisite flow rate, pressure, and volume so that the therapeutically effective amount is deliverable to the tumor target, or any combination of such vessels, conduits, or systems. For example, in embodiments, a nitric oxide generator can be used in whole or in part as a source of gNO; in other embodiments, a portable cylinder containing gNO can be used as the source; in yet other embodiments, a conduit delivering piped-in gNO from a distant storage facility can be used as the source. Combinations of sources can be used simultaneously or sequentially to provide the therapeutically effective amount of gNO. In an embodiment, a source of therapeutic gNO can be, for example, a NO gas cylinder or a NO generator as described in U.S. Pat. No. 9,573,110, the contents of which are included herein by reference; in a preferred embodiment, a NO gas cylinder is used, which can optionally be of a size and shape to permit ready portability. As used herein, the term “gas collector” refers to any receptacle, conduit, arrangement of filters, supports, adsorbers, absorbers, and the like, or other assemblies, mechanisms, pathways, or reactants that effect the removal of the spent gas, excess gas, residual gas, or byproducts of any of the foregoing that may be scavenged from the treatment region, and/or that absorb, adsorb, or react with any of such gases so that they are rendered chemically inactive or otherwise unavailable for further reactions. In embodiments, a gas collector can provide a chamber for collecting and retaining scavenged gas, which can be further equipped with reactants that adsorb, adsorb, or complex with the gas, such as activated coke or activated charcoal, silica gel, zeolites, soda lime, and the like. The gas collector can be dimensionally adapted for direct attachment to the delivery device, or for attachment to the delivery device via a conduit so that the gas collector can be positioned at a convenient location at a distance from the delivery device itself. The gas collector can include ancillary mechanisms for treating the scavenged gas or for adjusting its temperature, pressure, flow rate, or other physical parameters; one or more of such ancillary mechanisms can be combined with the gas collector itself to form a subsystem (an “evacuation subsystem”) of the overall gNO treatment system for evacuating the scavenged gas.

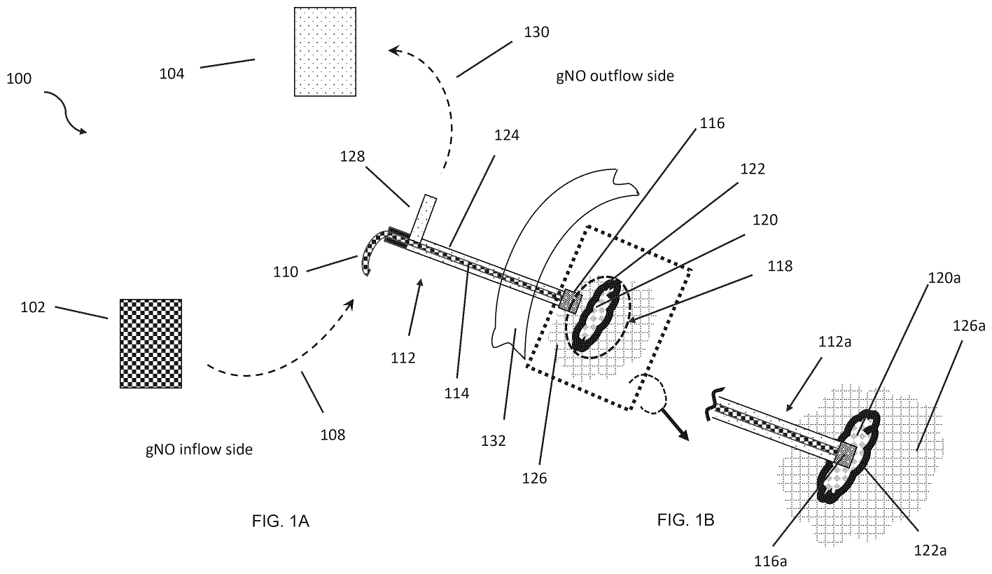

A general representation of a gNO treatment system in accordance with these systems and methods is depicted in FIG. 1A and FIG. 1B. FIG. 1A shows a schematic gNO treatment system 100 comprising a nitric oxide source 102 and a gas collector 104. The nitric oxide source 102 produces a gas inflow stream 108 of gNO that enters the gas inflow inlet 110 of the delivery device 112. The delivery device 112, to be described in more detail below, includes a NO inflow channel 114 in fluid communication with the gas inflow inlet 110, to direct the gNO into the tumor target region 118. The tumor target region 118 includes the tumor mass itself 120 and the circumjacent region 122 in therapeutic proximity to the tumor mass 120 within which the gNO can exert a therapeutic effect on the tumor mass 120. The tumor target region also includes the interior of the tumor mass, as illustrated in FIG. 1B and as described below in more detail. The delivery device 112 further includes a selectively permeable membrane 116, as will be described in more detail below, that is adapted both for gas delivery to and for gas collection from the tumor target region 118.

The delivery device 112 also includes a gas return channel 124 in fluid communication with the selectively permeable membrane 116. The selectively permeable membrane 116 permits the passage of the gNO into the tumor target region 118 (whether external to the tumor or internal to the tumor mass 120), and further permits scavenging of any gNO, whether such gNO has been delivered into the tumor target region 118 or whether such gNO has been delivered into the selectively permeable membrane 116 but has not passed into the tumor target region 118. Any scavenged gNO enters the gas return channel 124. The gas return channel 124 is in fluid communication with a gas return outlet 128 that passes a gas return stream 130 to the gas collector 104. While both the gas inflow stream 108 and the gas return stream 130 are represented in FIG. 1A by dashed directional arrows, it is understood that both gas inflow and gas return involve the passage of the gNO through appropriate conduits and valves (not shown), as would be understood by artisans of ordinary skill. It is further understood that the gas inflow stream 108 and gas return stream 130 can be monitored by appropriate pressure sensors, flowmeters, feedback systems and other surveillance and control equipment (not shown), as would be understood by artisans of ordinary skill.

The delivery device 112 is shown in FIG. 1A as penetrating a body wall 132, with the tumor target region 118 situated internal to the body wall 132. In embodiments, the tumor mass 120 is surrounded by normal tissue 126, which can be penetrated or pushed aside by the delivery device or by other surgical or endoscopic instruments in order to position the selectively permeable membrane 116 appropriately. In other embodiments, the tumor mass 120 is relatively accessible, without surrounding normal tissues 126 that require repositioning. The tumor target region 118 as depicted includes a tumor mass 120 and a surrounding mass of normal tissue 126; as shown, the delivery device is transgressing the normal tissue 126 to attain therapeutic proximity to the tumor mass 120. In the depicted embodiment, the selectively permeable tip 116 is close to, but not in contact with, the tumor mass 120. In other embodiments, the selectively permeable tip is in close proximity to the tumor mass 120, for example in the circumjacent region 122. In yet other, preferred, embodiments, the selectively permeable membrane 116 is in contact with the surface of the tumor mass 120, or actually penetrates it, as illustrated in more detail in FIG. 1B.

As shown in FIG. 1B, a selectively permeable membrane 116a can penetrate a tumor mass 120a, allowing the therapeutic dose of gNO to be delivered within the tumor mass 120a itself. As shown in this figure, the tumor mass 120a and circumjacent region 122a are surrounded by normal tissue 126a. In FIG. 1B, there is no tumor target region comparable to that shown in FIG. 1A, because the selectively permeable membrane 116a of the delivery device 112a is shown as penetrating the tumor mass 120a itself, and the tumor mass 120a thus constitutes the tumor target.

In the embodiments depicted in FIGS. 1A and 1B, the selectively permeable membranes 116 and 116a provide for gas delivery and for gas scavenging. In embodiments, the scavenged gas comprises gas that exits the selectively permeable membrane 116 or 116a and enters the tumor target area 118 and/or the tumor mass 120 or 120a itself. In other embodiments, and preferably, the scavenged gas is gas that remains in a region distal to the gas inflow channel 114 without passing through the selectively permeable membrane 116 or 116a to enter the tumor mass 120 or 120a and/or the tumor target area 118. This arrangement, where excess undelivered gNO is scavenged, is especially suitable for gas deployment within the tumor mass 120a (as shown in FIG. 1B), or into tissues immediately surrounding the tumor mass 120 or 120a where there is minimal free space for gas inflow. In these situations, the retrieval of undelivered gas can balance the outflow of gas into the tumor mass 120 or 120a or into the normal tissues 126 or 126a surrounding the tumor mass 120 or 120a. With the selectively diffusible properties of the membrane 116 or 116a, a high concentration (e.g., ≥1,000 ppm) of gNO can be directed at the tumor with diffusion of the gas into the tumor mass 120 or 120a or tumor target region 118, and with simultaneous or sequential removal of excess delivered gas or spent gas so that the residual gNO (or its byproducts) does not enter the tissues adjacent to the membrane 116 or 116a. The control over total gas efflux provided by the selectively permeable membranes 116 and 116a thus permits exposing the tumor target to the therapeutically effective amount of gNO while minimizing the deleterious effects of the gNO on other surrounding tissues.

It is understood that the delivery devices 112 and 112a and their selectively permeable membranes 116 and 116a, respectively, can be positioned using the entire range of techniques for accessing a tumor mass 120. In embodiments, the tumor mass 120 or 120a can be located within a body cavity, and the delivery device 112 can be inserted through the body wall 132 to access it. The insertion process can take place using endoscopic techniques familiar in the art. The insertion process can also take place using open techniques, wherein the tumor mass is accessed using conventional surgical approaches or less invasive surgical approaches, including without limitation full or limited laparotomy, thoracotomy, craniotomy, laminectomy, arthrotomy, and open retroperitoneal access procedures, and minimally invasive procedures such as, without limitation, laparoscopy, thoracoscopy, bronchoscopy, cystoscopy, gastroscopy, arthroscopy, hysteroscopy, colonoscopy, colonoscopy, sigmoidoscopy, and the like. In the depicted embodiment, with the tumor target 118 and/or tumor mass 120 or 120a covered by or buried in normal tissue 126 and 126a, open surgical techniques or less invasive surgical procedures can afford advantageous access. In other embodiments, the tumor target 118 and/or tumor mass 120 and 120a can be localized using radiographic or fluoroscopic procedures, with a needle placed to localize the lesion, following which the delivery device 112 and 112a can be directed into therapeutic proximity to the tumor target 118 and/or tumor mass 120 and 120a using radiographic or fluoroscopic procedures, as would be appreciated by artisans of ordinary skill in the field.

In embodiments, the delivery device 112a itself can be equipped with an introducer mechanism (not shown) that would allow the device 112a to penetrate the tumor mass 120a itself without requiring other surgical interventions. In embodiments, the introducer can be configured as an external cylindrical housing with a retractable blunt tip, wherein the blunt tip of the introducer penetrates the tumor mass and is then retracted to expose the selectively permeable membrane 116a. Other introducer mechanisms allowing the minimally invasive insertion of the device 112a into the tumor mass can be envisioned with no more than routine experimentation. In other embodiments, the device 112a can be constructed so as to facilitate more complete penetration and treatment of the tumor mass. For example, the tip of the device 112a can itself be pointed so that it penetrates the tumor mass, with openings (not shown) along the side of the tip structure for gas delivery and gas scavenging. As another example, the distal end of the device 112a can support a plurality of tips with corresponding membranes (not shown), each with openings for gas delivery and gas scavenging positioned at their ends or along their sides, to permit treatment of multiple sites within the tumor, either simultaneously or sequentially. In yet other embodiments, once the delivery device 112 or 112a is positioned in sufficient proximity to the tumor target, its distal end or its one or more tip structures can be separately manipulable, and can be directed more precisely to access the tumor target 118 and/or tumor mass 120 and 120a.

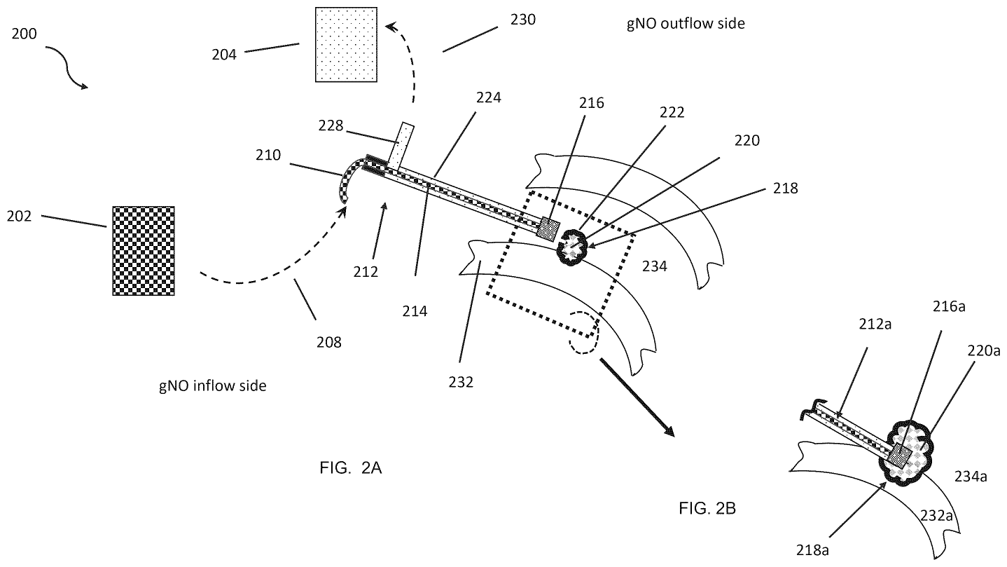

FIGS. 2A and 2B show a schematic gNO treatment system in accordance with these systems and methods. FIG. 2A shows a gNO treatment system 200 comprising a nitric oxide source 202 and a gas collector 204. The nitric oxide source 202 produces a gas inflow stream 208 of gNO that enters the gas inflow inlet 210 of the delivery device 212. The delivery device 212 includes a gNO inflow channel 214 in fluid communication with the gas inflow inlet 210, to direct the gNO into the tumor target region 218. The tumor target region 218 includes the tumor mass itself 220 and the circumjacent region 222 in proximity to the tumor mass 220 within which the gNO can exert a therapeutic effect on the tumor mass 220. The delivery device further includes a selectively permeable membrane 216, as will be described in more detail below, that is adapted both for gas delivery to and for gas collection from the tumor target region 218. The delivery device 212 also includes a gas return channel 224 in fluid communication with the selectively permeable membrane 216. The selectively permeable membrane 216 permits the passage of the gNO into the tumor target region 218 (whether it is positioned external to the tumor or internal to the tumor mass 220), and further permits scavenging of any gNO, whether such gNO has been delivered into the tumor target region 218 or whether such gNO has been delivered into the selectively permeable membrane 216 but has not passed into the tumor target region 218. Any scavenged gNO enters the gas return channel 224. The gas return channel 224 is in fluid communication with a gas return outlet 228 that passes a gas return stream 230 to the gas collector 204. While both the gas inflow stream 208 and the gas return stream 230 are depicted in FIG. 2A as dashed directional arrows, it is understood that both gas inflow and gas return involve the passage of the gNO through appropriate conduits and valves (not shown), as would be understood by artisans of ordinary skill. It is further understood that the gas inflow stream 208 and gas return stream 230 can be monitored by appropriate pressure sensors, flowmeters, feedback systems and other surveillance and control equipment (not shown), as would be understood by those of ordinary skill in the art.

The delivery device 212 is shown in FIG. 2A as positioned within a body lumen 234, and the tumor mass 220 is shown as attached to, arising from, or embedded in one of the walls 232 of the lumen 234, so that it is accessible by an intraluminal approach. While the embodiment in FIG. 2A shows a gap between the selectively permeable membrane 216 and the tumor target region 218, it would be preferable for the gap between these two structures to be minimized, so that the gNO is not delivered freely into the lumen 234. In a preferred embodiment, the selectively permeable membrane 216 contacts the tumor mass 220 directly. In an even more preferred embodiment, the selectively permeable membrane is positioned as shown in FIG. 2B, with such membrane 216a embedded in or wholly or partially surrounded by the tissue of the tumor mass 220a, so that the gNO is delivered into the tumor mass 220a without extravasation into the lumen 234a. The embodiment in FIG. 2B shows a tumor target region 218a surrounding the tumor mass 220a; delivery of gNO into the tumor target region 218a can have a therapeutic effect on the tumor mass 220a even if the tumor mass 220a is not penetrated by the delivery device 212a. Although delivery of gNO within the tumor mass 220a is preferred, it is understood that penetrating the tumor mass 220a through an intraluminal approach can be challenging, especially if the tumor mass 220a is pedunculated or otherwise poorly tethered to the wall 232a of the lumen 234a.

In the embodiments depicted in FIGS. 2A and 2B, the selectively permeable membranes 216 and 216a provide for gas delivery and for gas scavenging. In embodiments, the scavenged gas comprises gas that exits the selectively permeable membranes 216 or 216a and enters the tumor target area 218 and/or the tumor mass 220 or 220a itself. In other embodiments, and preferably, the scavenged gas is gas that remains in a region distal to the gas inflow channel 214 (not shown in FIG. 2A) without passing through the selectively permeable membrane 216 and 216a to enter the tumor mass 220 or 220a and/or the tumor target area 218 or 218a. This arrangement, where excess undelivered gNO is scavenged, is especially suitable for gas deployment within the tumor mass 220a (as shown in FIG. 2B) or within the tumor target area 218 and 218a, or in situations in which the selectively permeable membrane 216 or 216a is in direct contact with the tumor mass 220 or 220a or the tumor target tissues 218 or 218a immediately adjacent to the tumor mass 220 or 220a. In these situations, there is minimal free space for gas flow into the lumen 234 or 234a, or into the healthy tissues of the lumen wall 232 or 232a.

Retrieving the undelivered gas in these situations gas can balance the outflow of gas into the tumor mass 220 or 220a (for example, the diffusion of the gas into the tumor mass) or into the normal tissues of the tissue target region 218 or 218a, and can further prevent delivery of excess gas into the lumen 234 or 234a. With the selectively diffusible properties of the membrane 216 or 216a, a high concentration (e.g., ≥ 1,000 ppm) of gNO can be directed at the tumor with diffusion of the gas into the tumor mass 220 or 220a or tumor target region 218 or 218a, and with simultaneous or sequential removal of excess delivered gas so that the excess does not enter lumen 234 or 234a or the healthy tissues adjacent to the membranes 216 or 216a. The control over total gas efflux provided by the selectively permeable membrane 216 or 216a thus permits exposing the tumor target to the therapeutically effective amount of gNO while minimizing the deleterious effects of the gNO on other surrounding tissues.

It is understood that the delivery devices 212 and 212a can be positioned within the lumens 234 and 234a, respectively, using the entire range of techniques for accessing a tumor mass 220 or 220a within such lumen 234 or 234a. An intraluminal-accessible lumen 234 can be any cavity or channel within an organ or anatomic structure, such as, without limitation, the interior cavity of the uterus, the bladder, the stomach, the gall bladder, the nasopharynx, the oropharynx, and the like, and the interior channel of the urethra, the colon, the bronchi, the trachea, a blood vessel, and the like. For purposes of illustration, the term “lumen” can be applied to all cavities or channels within organs or anatomic structures and all potential cavities or channels in such organs and anatomic structures, such as the interior cavity of a joint or the interior cavity of the cerebral ventricles, or the intrapleural space, the retroperitoneal space, the subdural space, the subarachnoid space, and the like. It is understood that access to body lumina using minimally invasive techniques is familiar to those of ordinary skill in the art.

Once the delivery device 212 or 212a is positioned in sufficient proximity to the tumor target 218 or 218a, in embodiments its distal end is separately manipulable, and can be directed more precisely to access the tumor target 218 or 218a. With or without separate manipulation of the distal end of the delivery device 212a, the insertion of the selectively permeable membrane 216a into the tumor mass 220a can be accomplished by standard techniques familiar to artisans of ordinary skill. In embodiments, the device 212a can be constructed so as to facilitate more complete penetration and treatment of the tumor mass. For example, the tip of the device 212a can itself be pointed so that it penetrates the tumor mass, with openings (not shown) along the side of the tip structure for gas delivery and gas scavenging. As another example, the distal end of the device 212a can support a plurality of tips with corresponding membranes (not shown), each with openings for gas delivery and gas scavenging positioned at their ends or along their sides, to permit treatment of multiple sites within the tumor, either simultaneously or sequentially. In yet other embodiments, once the delivery device 212 and 212a is positioned in sufficient proximity to the tumor target, its distal end or its one or more tip structures can be separately manipulable, and can be directed more precisely to access the tumor mass 220 and 220a.

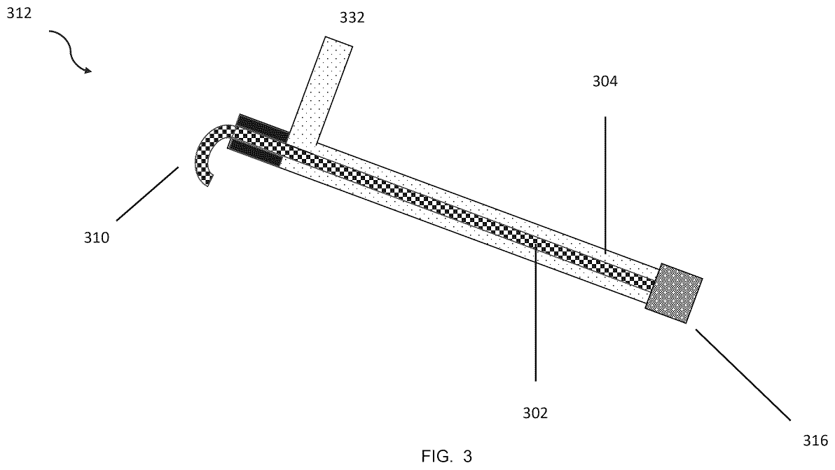

In more detail, a delivery device for use with these systems and methods is shown schematically in FIG. 3. As shown in FIG. 3, a delivery device 312 is adapted for use with a delivery system (as shown schematically in the previous FIGS.) for delivery of gNO to a tumor target. The delivery device 312 shown in this figure is used for the insufflation of gNO and for its scavenging after delivery. Accordingly, the device 312 comprises a hollow insufflation member that confines an insufflation channel 302 and a hollow gas return sleeve that confines a gas return channel 304, with the insufflation member and its enclosed insufflation channel 302 coaxially arranged with respect to the gas return sleeve and its enclosed gas return channel 304. As shown in this figure, the insufflation member defining the insufflation channel 302 is confined within the outer gas return sleeve and its enclosed gas return channel 304, although in other embodiments, the gas return channel 304 can instead be positioned within an outer insufflation channel 302. In embodiments, the gas return channel 304 can be a single channel that circumscribes the insufflation channel 302, while in other embodiments, a plurality of gas return channels 304 can be positioned surrounding the insufflation channel 302. Similarly, in other embodiments, a plurality of insufflation channels 302 can be positioned surrounding the gas return channel 304. In embodiments, any combination of single or multiple insufflation channels 302 and gas return channels 304 within the delivery device can be employed in keeping with this disclosure. In the depicted embodiment, the inner insufflation channel 302 is positioned approximately centrally within the outer gas return channel 304, although other relative positioning variations can be envisioned in accordance with this disclosure. Similarly, while each channel can have a circular cross-section, with the space between them an annular space, any number of alternative geometries can be envisioned in accordance with this disclosure.

It is anticipated that the delivery device 312 is advanced to contact a tumor target or to penetrate the tumor target using conventional open surgical techniques or endoscopic techniques. If open surgical access to the tumor target is performed, the delivery device 312 itself will not need to perform dissection or penetrate the target tissues or their surroundings. Similarly, using endoscopic techniques, the delivery device 312 in embodiments, can be positioned in sufficient proximity to the tumor target that further tissue dissection or penetration is unnecessary, while in other embodiments, the positioning of the device 312 can be preceded by or accompanied by dissection to permit its access to the tumor target or its penetration of the tumor target or tumor mass. In situations where the delivery device 312 also serves as a probe to create a passageway through tissues or tissue spaces, the delivery device 312 can be surrounded by a protective sheath (not shown), which can cover the tip of the delivery device 312 during its insertion and advancement, and which can be retracted along the axis of the device 312 when the device 312 has reached the tumor target. In embodiments, a protective sheath can have a rounded tip to facilitate passage of the device 312 through tissues and potential spaces in the body. In embodiments the distal tip itself can be constructed so that it can penetrate both normal and abnormal tissues, and penetrate the tumor mass. For example, the tip can be pointed or otherwise sharpened to allow tissue penetration. In such an embodiment, the one or more openings in the tip to permit gas deliver and gas scavenging can be positioned on the pointed or sharpened part of the tip, or along its sides, or both. As another example, the distal end of the device can support a plurality of tips (not shown), each with openings for gas delivery and gas scavenging positioned at their ends or along their sides, to permit treatment of multiple sites within the tumor, either simultaneously or sequentially. In certain embodiments, once the distal end of the delivery device is positioned in sufficient proximity to the tumor target, its distal end or its one or more tip structures can be separately manipulable, and can be directed more precisely to access the tumor mass.

In embodiments, the delivery device 312 can incorporate a channel (not shown) for an endoscope, which can monitor the positioning of the device. In other embodiments, the device 312 can be advanced through the end of an endoscope so that the insertion of the device 312 into the body, the advancement of the device 312 towards the tumor target, and the retraction of the device 312 from the body can be observed visually or virtually, or otherwise can be tracked by those individuals performing the overall treatment procedure. During an endoscopic procedure, the positioning of the device 312 can be accomplished by a single instrument that permits both the positioning of the device 312 and its direct visualization. Alternatively, endoscopic techniques can permit the installation and positioning of the device 312 using one instrument, with the observation of positioning being performed by a different instrument. The delivery device 312 can be employed in conjunction with other instrumentation for dissection, tissue exposure, irrigation, suction, cauterization, tissue sampling, and the like, as would be understood by artisans of ordinary skill. Direct visual observation is not required for effective positioning and operation of the device: for example, the positioning of the device 312 can be monitored fluoroscopically, or its position can be directed robotically and/or in accordance with stereotactic positioning information generated by and processed by a computer.

As mentioned above, the device 312 is inserted into the patient's body and is advanced towards the tumor target, with its selectively permeable diffusion membrane 316 at its tip being positioned in proximity thereto or being inserted into the tumor mass in whole or in part. When the device 312 is appropriately positioned, gNO is supplied through the insufflation channel 302 to exit the distal end of the device 312. The distal end (not shown) of the insufflation channel 302 can be shaped in any way that facilitates and optimizes delivery of the gNO. For example, the distal end can be shaped as a simple, flat-ended cylinder, permitting a uniform dispersion of the gNO over the area to be treated. In embodiments, the distal end of the insufflation channel can be shaped as a nozzle or covered with a perforated membrane or otherwise, in order to shape and direct the flow of gNO, or to adjust the velocity of the gas flowing from the distal end, as would be envisioned by skilled artisans. In embodiments, the distal end of the insufflation channel 302 extends beyond the distal end of the gas return channel 304, while in other embodiments, the distal end of the gas return channel 304 extends beyond the distal end of the insufflation channel 302. In embodiments where the distal end of the insufflation channel 302 extends beyond the distal end of the gas return channel 304, the openings for gas delivery can be positioned on the side of the distal end of the insufflation channel in addition to or instead of on the distal end. In such embodiments, the distalmost end of the insufflation channel can be sealed or formed as a blunt or sharpened structure that can be used for penetrating tissues, with gas delivery taking place through openings on the side of the structure in addition to or instead of through an opening on the distal end. In yet other embodiments, the distal end of the insufflation channel and the distal end of the gas return channel are approximately even with each other. In any of the foregoing embodiments, when the device 312 is appropriately positioned with the selectively permeable diffusion membrane 316 in proximity to the tumor target or embedded in whole or in part into the tumor target, gNO is directed from the distal end of the device 312 into the tumor target.

The delivery device 312 is in fluid communication with a gas inflow source (not shown) that provides a gas inflow stream (not shown) that enters the delivery device 312 through a gas inflow inlet 310. Gas delivery can be regulated locally, through controls on the delivery device 312 itself. For example, the distal end of the insufflation channel 302 can be equipped with a variable seal (not shown) or diaphragm that can be adjusted to close off the channel 302 entirely, or open up the channel 302 to permit the selected amount of gNO to be delivered to the tumor target through an aperture in the diaphragm of a selected size. Such an arrangement permits the delivery device 312 to be advanced to the target tissue, with the volume and flow of gNO delivery to be adjusted by a mechanism on the device itself, permitting fine-tuning of gNO delivery at the delivery site. In other embodiments, the volume and flow of gNO delivery is coordinated more proximally, for example by regulating the passage of gNO through the gas inflow inlet 310, or by regulating the amount of gas delivered into the system from the gas inflow source (not shown) or by regulating its passage through the conduits (not shown) connecting the gas inflow source to the gas inflow inlet 310.

The delivery device 312 further includes a gas return channel 304 for scavenging the gNO that has been directed at the tumor target. In an embodiment, after the tumor target has been exposed to the preselected amount of gNO, the gNO and its byproducts are recovered or scavenged by the delivery device 312; in an alternative embodiment, the scavenging proceeds simultaneously with the exposure of the tumor target to the gNO, so that any excess or spent gNO or byproducts thereof are removed expeditiously. In a preferred embodiment, the scavenging serves to remove unused gNO from the delivery area enclosed by the selectively permeable diffusion membrane 316; the delivery of gNO can thus be balanced with scavenging unused or excess gNO before it reaches or enters the target tissues. In this way, the appropriate amount of gNO is delivered into the target tissues, for example by diffusion into the tissues in proximity to or in contact with the selectively permeable diffusion membrane 316, with the spent or excess gas being removed before it has the opportunity to reach the tissues. In embodiments, a sensor system can be incorporated in the delivery device 312 that can balance the amount of gNO being delivered to the target tissue (e.g., by measuring the flow or volume delivered of gas with a known nitric oxide concentration) versus the amount scavenged, to determine the dose of gNO delivered to the target tissue and compare that dose to the therapeutically effective amount intended for the target tissue. In embodiments, the sensor system can be integrated with computer-based or other automatic controls that regulate the inflow amount, flow rate, duration of delivery, or timing of delivery of gNO to the target tissue, based on feedback information provided by one or mor sensors in the sensor system. For example, the computer-based or other automatic controls can alter the inflow or outflow parameters, or can alter the pressure in the gas return channel 304 (for example by reducing the pressure or by applying an intermittent vacuum or partial vacuum in that channel) so as to increase the amount of extra, residual, or spent gNO that is scavenged. In preferred embodiments, the delivery of gNO to the tissues (for example by diffusion into the tissues in proximity to or in contact with the selectively permeable diffusion membrane 316) and the scavenging are performed simultaneously, to prevent overexposure of the target tissues and normal tissues to the gNO. In other embodiments, the delivery of gNO is followed by a scavenging operation.

The distal end of the gas return channel 304 can be designed to facilitate or optimize the removal of excess gas, residual gas, spent gas, and byproducts thereof (collectively, “recovered gases”). For example, the distal end can be flared so that it can address a larger area for removing recovered gases, including gNO that extravasates from the intended treatment area as well as gNO that has not been delivered into the treatment area. In embodiments, the flaring of the distal end can be adjustable, allowing the distal end to be expanded or extended in response to the therapeutic delivery of the gas, and/or to conform to the size and shape of the area exposed to the gNO. Other factors can be considered in designing the shape and adjustability of the distal end of the gas return channel 304, as would be appreciated by artisans of ordinary skill in the field. The distal end of the gas return channel 304 can extend out beyond the distal end of the insufflation channel 302, or vice versa, or the two distal ends can be approximately level with each other.

The gases scavenged through the distal end of the gas return channel 304 is returned proximally through this channel in the delivery device 312, to pass through the gas return outlet 332 that is in fluid communication with the gas collector (not shown) of the overall delivery system, as has been shown schematically in previous Figures. In embodiments, gas return can take place by diffusion into the gas return channel 304 of excess gas delivered from the insufflation channel 302, where the excess gas is confined within the selectively permeable diffusion membrane 316 (described in more detail below) without encountering external tissues or structures. In other embodiments, gas return can take place by diffusion into the gas return channel of gas that has already passed through the selectively permeable diffusion membrane 316 into surrounding tissues, where such gas is in excess amount or where such gas has extravasated from the intended therapeutic region. In either case, the excess or spent gNO (or their byproducts) so removed passes proximally through the gas return channel 304, ultimately to enter the gas collector, as depicted in previous Figures. The proximally-directed passage of the scavenged gas can take place by diffusion, and/or can be caused by or expedited by a pressure differential, for example a lower pressure environment such as a partial vacuum created in the gas return channel 304 or its more proximal connecting components. In an embodiment, the gas collector comprises a gas evacuation subsystem. A gas evacuation subsystem can include additional mechanisms or processes that cause, facilitate, or expedite the return of recovered gas. For example, in an embodiment, a gas evacuation subsystem can form an appropriate lower pressure environment intended to cause, facilitate, or expedite the return of the recovered gas from the region surrounding the tumor target, so as to minimize the local effects of the gNO or its byproducts on the healthy tissue surrounding the tumor target. In another embodiment, the gas evacuation subsystem can form an appropriate lower pressure environment intended to cause, facilitate, or expedite the return of excess unused gas simultaneously or near-simultaneously with its delivery, so that the excess gas does not pass through the selectively permeable diffusion membrane 316 to reach the surrounding tissues. Other gas evacuation subsystems can be envisioned by skilled artisans with the aim of causing, facilitating, or expediting the return of the recovered gases from the tumor target itself or the area surrounding it.

In the depicted embodiment, the selectively permeable diffusion membrane 316 envelopes the distal ends of both the insufflation channel 302 and the gas return channel 304. In an embodiment, the diffusion membrane 316 permits the insufflation of gNO into the tumor target region and also permits the scavenging of gNO from the tumor target region and surroundings, so that the intended cytotoxic effect of the gNO on the tumor mass is controlled and so that the unintended cytotoxic effect of the gNO on the surrounding tissues is minimized. In another embodiment, the diffusion membrane 316 selectively restricts the outflow of gNO towards the tissues to permit the simultaneous or near-simultaneous recovery of excess gNO, allowing the appropriate amount of gNO to be directed into the tissues in order to have the therapeutic effect on the tumor mass. Selection of an appropriate material for the diffusion membrane 316 can be based on factors familiar to skilled artisans, for example the diffusion coefficient of the various materials, as described in Mowery et al., Polymer 40 (1999) 6203-6207, and the gNO release profiles of the materials, as described in Ren et al., ACS Biomater. Sci. Eng. 2 (2016) 1483-1492; the contents of both these publications are incorporated herein by reference in their entirety. Materials can include plasticized and unplasticized polymeric films such as silicone rubbers, polyurethanes (including without limitation aliphatic polyurethanes, aromatic polyether polyurethanes, silicone and/or polycarbonate-containing polyurethanes, and the like), polyvinylchlorides, fluoropolymers (such as polytetrafluoroethylene and the like), cellulose triacetates, polysulfones, and copolymers and mixtures thereof, or other similar materials. Examples of suitable materials are provided in Table S1 the Supplemental Materials (incorporated by reference herein in its entirety) for the Ren publication cited above, which can be accessed at the following site: pubs.acs.org/doi/abs/10.1021/acsbiomaterials.6b00215 (accessed Mar. 13, 2022).

In embodiments, the diffusion membrane 316 is constructed from a single polymeric material; in other embodiments, the diffusion membrane 316 can be composed of different polymeric segments, for example to provide one set of diffusion properties for the insufflation region of the diffusion membrane 316 versus those for the scavenging region of the diffusion membrane. In embodiments, the diffusion membrane 316 can be physically segmented (not shown), so that a first diffusion membrane segment covers the distal end of the insufflation channel 302 and any gas access space solely in fluid communication therewith and a second diffusion membrane segment covers the distal end of the gas return channel 304 and any gas access space solely in fluid communication therewith. Each segment can possess similar or different diffusion parameters to allow different permeabilities, whereby gNO outflow responds to one set of diffusion parameters, and spent or excess gas scavenging responds to a different set of diffusion parameters. Such an arrangement can advantageously permit the scavenging function to include both excess gNO delivered from the insufflation channel 302 and excess or spent gas that has reached the tissues outside the diffusion membrane 316.

In embodiments, the diffusion membrane 316 can include a plurality of pores, wherein the size of the pores can range from nanometers to micrometers. In an embodiment, in order to selectively allow gNO to pass through the diffusion membrane 316 while simultaneously blocking NO₂, the pore size should be smaller than the size of a NO₂ molecule, e.g., 0.1197 nm in diameter, but larger than the size of an NO molecule, e.g., 115 nm in diameter. The pore sizes of the diffusion membrane 316 can be adjusted by changing the synthesis conditions of the membrane, such as the temperature, solvent composition, and polymer concentration. The membrane thickness and composition can also be optimized to achieve the desired selectivity based on ionic characteristics. Further, the membrane thickness, operating pressure, and gas flow rate can also affect the effectiveness of the membrane for gas separation.

In more detail, as shown in FIG. 3, the diffusion membrane 316 covers the distal tip of the delivery device 312, with the distal tip structures comprising the diffusion membrane 316, a gas access space enclosed by the diffusion membrane 316, the distal end of the insufflation channel 302 and the distal end of the gas return channel 304. The diffusion membrane 316 is dimensionally adapted to cover the distal end of both channels, and thus can be configured to conform to the shape of both distal ends. For example, if the distal end of the gas return channel 304 is flared to permit gas scavenging over a wider area, the diffusion membrane can be flared as well. A support (not shown) for the diffusion membrane 316 can be provided as a distal tip structure, so that the diffusion membrane 316 is positioned at an appropriate distance from the distant ends of the insufflation channel 302 and the gas return channel 304 to create a space into which the gas flowing out into the tissues can pass, and into which the gas being scavenged can return. The positioning of the diffusion membrane 316 can create one or more gas access spaces (not shown) for gas delivery, gas return or both, with the one or more gas access spaces being in fluid communication with the insufflation channel 302, the gas return channel 304 or both.

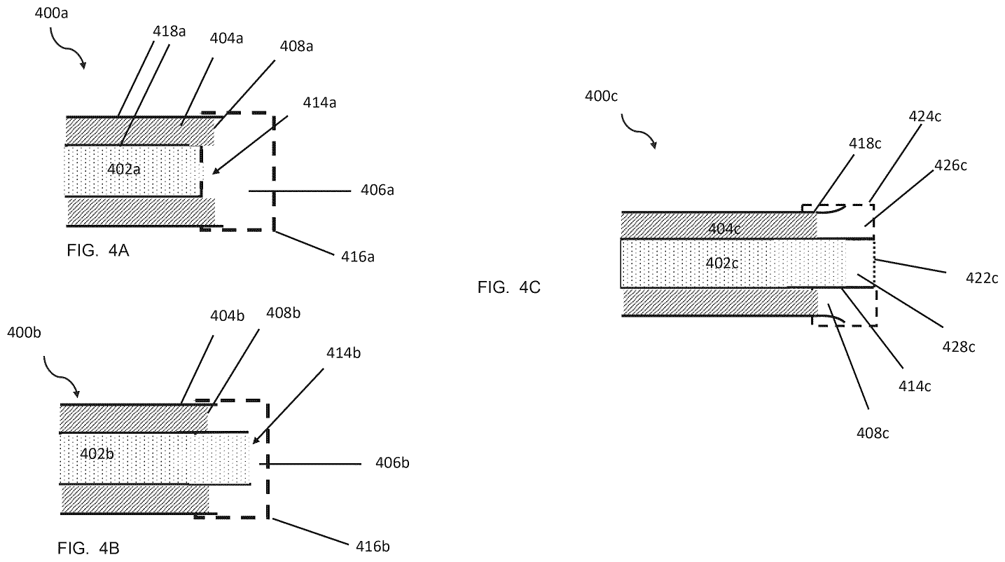

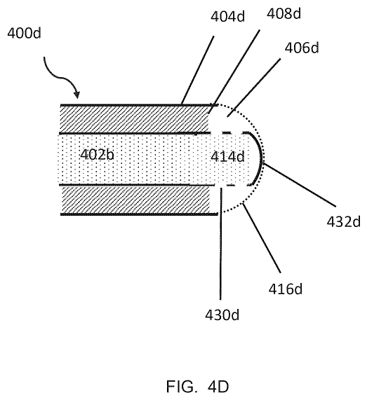

FIGS. 4A and 4B depict schematically embodiments of the distal portion 400a and 400b of a delivery device in accordance with these systems and methods, showing in more detail embodiments of the distal tip structure as described above with certain of its components: one or more diffusion membranes, one or more gas access spaces, the distal end of the insufflation channel, and the distal end of the gas return channel. Each of these distal tip structures is described below with reference to FIGS. 4A and 4B.

As shown in FIG. 4A, the distal portion 400a of the delivery device is configured for both delivery of gNO to a tumor target (not shown) and scavenging of gNO. In embodiments, the scavenging phase of the process can take place simultaneously with the delivery phase, so that excess gNO is removed and/or spent gNO is removed at the same time that gNO is being directed and diffusing into to the tumor target itself. In addition or alternatively, the scavenging phase of the process can take place following the delivery phase, so that some or all of the delivered amount is recovered or removed following delivery. FIG. 4A shows one exemplary arrangement for the distal portion 400a of the delivery device that permits both insufflation and scavenging. As shown in this figure, an insufflation channel 402a contains gNO to be delivered to the tumor target. The gNO can be directed through the distal end 414a of the insufflation channel 402a, to cross the shared gas access space 406a and reach the tumor target (not shown). As shown in this figure, the distal end 414a of the insufflation channel 402a has an optional constriction relative to the diameter of the insufflation channel 402a; in other embodiments, there is no constriction of this channel, or it is shaped in a different way; it would be understood by skilled artisans that the dimensions and shape of the distal end 414a of the insufflation channel can be engineered to optimize the flow characteristics and/or velocity of the gNO stream being directed at the tumor target. The shared gas space 406a is in fluid communication with the insufflation channel 402a and also with the gas return channel 404a. As shown in this figure, the gas return channel 404a opens into the shared gas access space 406a; any gas within the shared access space 406a can be retrieved into the gas return channel 404a through the distal end 408a of the gas return channel 404a. As shown in this figure, the distal end 408a of the gas return channel 404a extends beyond the distal end 414a of the insufflation channel 402a; it is understood that the relative positioning of the distal ends of the two channels can be engineered to optimize gas insufflation and gas retrieval, with the two ends being substantially coplanar, or with one protruding or recessed relative to the other. In embodiments, the relative positioning of the two distal ends can be adjusted dynamically, for example by retracting or extending one channel relative to the other. As shown in this figure, the walls 418a of the gas return channel 404a are collinear, neither flared nor tapered. In other embodiments, the walls 418a at the distal end 408a can be flared, tapered or otherwise engineered to optimize the gNO retrieval.

As shown in FIG. 4A, a diffusion membrane 416a covers the distal ends of both channels as a single unit. In embodiments, the diffusion membrane 416a can be made of a single homogeneous material; in other embodiments, the material can be heterogeneous, for example incorporating different polymeric substances in different regions. In the depicted embodiment, the diffusion membrane 416a is shown as a single structure that surrounds a single shared gas access space 406a. That shared gas access space 406a can be occupied by gNO intended for insufflation into the tissues, or gas being scavenged from the tissues, or both. In a preferred embodiment, excess gas being delivered into the shared gas access space 406a is scavenged simultaneously, near-simultaneously or sequentially through the distal end 408a of the gas return channel 404a, thereby preventing excessive gas delivery into the tissues. The diffusion membrane 416a circumscribes the shared gas access 406a and retains the delivered gNO therein to permit the recovery of any excess gNO, so that a therapeutically effective amount of gNO reaches the tumor target without a gas excess or spent gas residuum that can adversely affect the tumor target itself or the tissues surrounding it.

FIG. 4B presents an alternative embodiment of a distal portion 400b of a delivery device, in which the distal end 414b of the insufflation channel 402b extends beyond the distal end 408b of the gas return channel 404b. Both the insufflation channel 402b and the gas return channel 408b are in fluid communication with the shared gas space 406b, so that any gas delivered by the insufflation channel 402b into the shared access space 406b can be retrieved into the gas return channel 404b through the distal end 408b of the gas return channel 404b. As depicted, the gas delivered by the insufflation channel 402b enters the shared gas access space 406b through an opening at its distal end 414b. In other embodiments, there can be openings (not shown) in the sides of the insufflation channel 402b where the distal portion of the insufflation channel 402b projects into the shared gas access space 406b, so that gas delivered by the insufflation channel 402b passes into the shared gas access space 406b instead of or in addition to passing through an opening at the distal end 414b of the insufflation channel 402b. In certain embodiments, the distal end 414b can be sealed entirely, and can be formed as a closed structure configured as a blunt, rounded, or pointed tip, with gas entering the shared gas access space 406b only through the openings (not shown) in the sides of the insufflation channel 402b, as is shown in more detail in FIG. 4D. The diffusion membrane 416b extends beyond and covers or arches over the distal ends of both channels. As was previously described for FIG. 4A, the diffusion membrane 416b can be made of a single homogeneous material or can be heterogeneous, for example incorporating different polymeric substances in different regions. The diffusion membrane 416b is shown in FIG. 4B as a single structure that surrounds a single shared gas access space 406b. In embodiments, that shared gas access space 406b can be occupied by gNO intended for insufflation into the tissues, or can be occupied by gas being scavenged from the tissues, or both. In a preferred embodiment, excess gas being delivered into the shared gas access space 406b is scavenged simultaneously, near-simultaneously or sequentially through the distal end 408b of the gas return channel 404b, thereby preventing excessive gas delivery into the tissues. The diffusion membrane 416b circumscribes the shared gas access 406b and retains the delivered gNO therein to permit the recovery of any excess gNO, so that a therapeutically effective amount of gNO reaches the tumor target without a gas excess that can adversely affect the tumor target itself or the tissues surrounding it. In embodiments, the relative positioning of the distal end 414b of the insufflation channel 402b and the distal end 408b of the gas return channel 404b can be determined so that the gas outflow through the insufflation channel 402b into the surrounding tissues is balanced by the gas retrieval taking place through the gas return channel 404b. The selectively permeable diffusion membrane 416b can sufficiently restrict the gas outflow from the gas access space 406b into the surrounding tissues so that any excess gas that has been delivered into that space 406b can be retrieved, thereby preventing the excess gas from entering the surrounding tissues. While the embodiments depicted in FIGS. 4A and 4B show the distal ends of the insufflation channel and of the gas return channel at different levels relative to each other, with one extending in whole or in part distal to the other, in other embodiments not shown here the distal ends of the insufflation channel and the gas return channel can be substantially even with each other, so that one does not extend distal to the other and one is not recessed relative to the other.

FIG. 4C presents an alternative embodiment of a distal portion 400c of a delivery device in which separate diffusion membranes 422c and 424c are positioned over the distal ends of the insufflation channel 402c and the gas return channel 404c, respectively. The diffusion membranes 422c and 424c can be affixed directly to the walls of the distal end 408c and 414c of each channel, or one or both can be supported by a separate structure integrated with or separate from the walls of each channel (not shown). In the depicted embodiment, the walls 418c of the gas return channel 404c are shown as flared, although other design choices for the shape of the distal end 408c of the gas return channel 404c can be envisioned by those having ordinary skill in the art. In the depicted embodiment, separate gas access spaces 428c and 426c are available for the insufflation channel 402c and the gas return channel 404c, respectively. In the depicted embodiment, the distal portion 414c of inner insufflation channel 402c is shown as extending beyond the distal end 408b of the gas return channel 404b; it is understood that other relative positions of the two channels can be arranged, consistent with the systems and methods disclosed herein.

FIG. 4D presents an alternative embodiment of the distal portion 400d of a delivery device, in which the inner insufflation channel 402b has one or more openings 430d along the sides of the distal end 414d. The one or more openings 430d allow gas to be delivered from the insufflation channel 402d into the shared gas access space 406d; both the insufflation channel 402d and the gas return channel 408d are in fluid communication with the shared gas space 406d, so that any gas delivered by the insufflation channel 402d into the shared access space 406d can be retrieved into the gas return channel 404d through the distal end 408d of the gas return channel 404d. In the depicted embodiment, the gas delivered by the insufflation channel 402d enters the shared gas access space 406d through the one or more openings 430d on the sides of the distal end 414d that projects into the shared gas access space, while the distalmost portion of the distal end 414d is sealed off; it is understood, however, that there can be one or more openings in the distal end 414d in addition to the openings 430d on the sides of the distal end 414d. In the depicted embodiment, the sealed off distal end 414d has a rounded blunt tip, which can act to penetrate tissues in order to optimize positioning of the delivery device in proximity to the tumor target. In other embodiments, the tip can be sharpened or pointed instead of blunted. In the depicted embodiment, the diffusion membrane 416d encloses the shared gas access space 404, which is arranged as a toroid surrounding the central distal end of the insufflation channel 414d. The diffusion membrane 416d can be draped over the distal end 414d of the insufflation channel, and attached thereto, as depicted. In other embodiments, the diffusion membrane can be affixed to the distal end 414d more proximally, so that the blunt or pointed tip structure protrudes without any membranous covering; in such an embodiment, the blunt or pointed end can be used for dissection or tissue penetration with less risk of damaging the diffusion membrane 416d and inadvertently releasing the contents of the shared gas access space 406b.

While the foregoing tip structures in FIGS. 4A-D are described as individual structures, it is understood that any delivery device can be equipped with one or more tips, which can be the same or different from each other in construction. For example, a central tip similar to that of FIG. 4D can be included in a tip array, with other tips surrounding it that lack a blunt end; in such a configuration, the central tip can be used as an initial dissector of the tissues and tumor mass to create a central channel that facilitates the penetration of the other adjacent tips without blunt ends. As another example, a central set of tips can be equipped with openings at the distal ends of their insufflation channels to direct gas at the tumor target directly ahead, while a peripheral set of tips can be equipped with openings on the sides of their insufflation channels to direct gas at the tumor target lateral to the delivery device. Other arrangements can be envisioned by skilled artisans in the field.

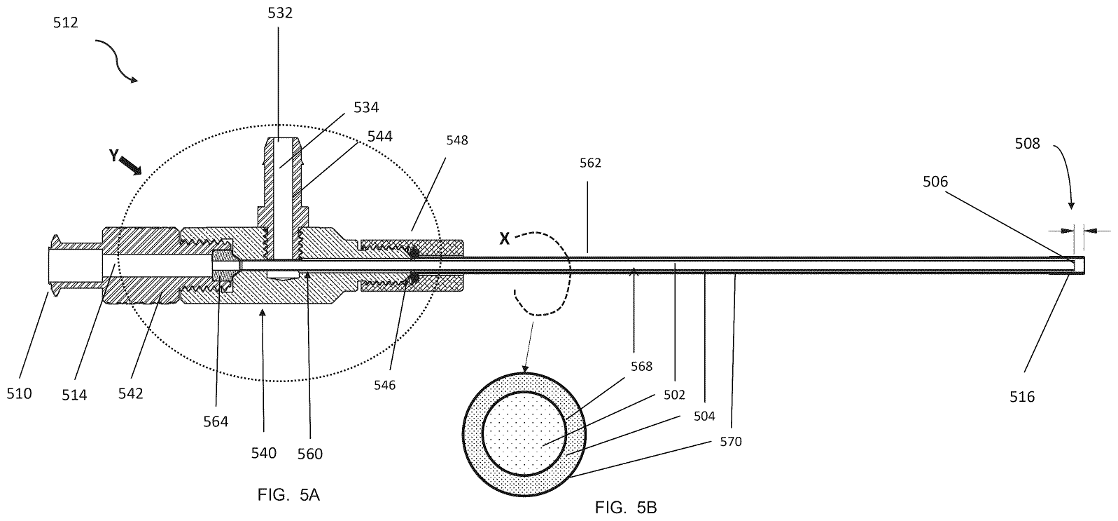

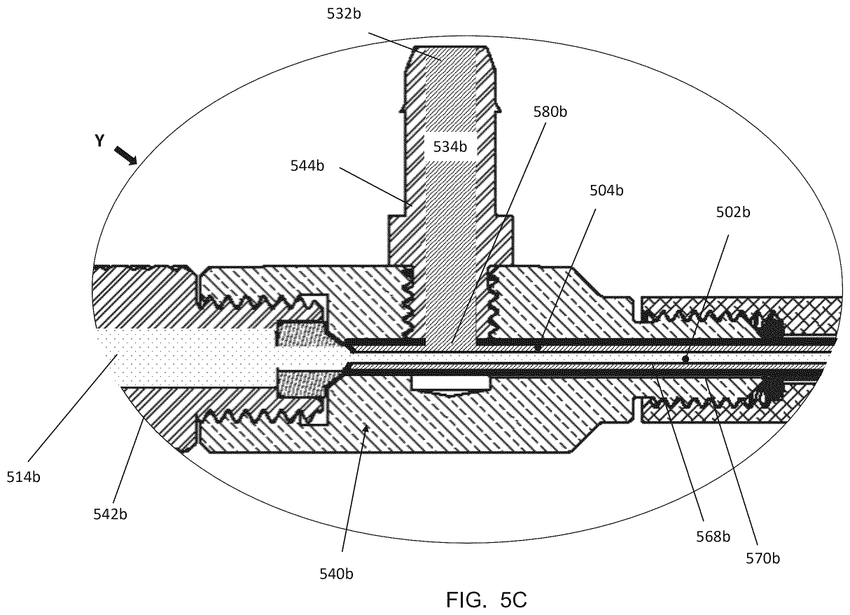

An embodiment of a delivery device in accordance with these systems and methods is depicted in longitudinal cross-section in FIG. 5A and FIG. 5B, where FIG. 5B represents a transverse cross-section of a component of the device as taken at dotted-line X. The delivery device 512 is adapted for use with an overall treatment system for providing gNO inflow and for retrieving scavenged gNO from the tumor target area. gNO inflow from the treatment system enters the delivery device 512 through a gas flow inlet 510, which is in fluid communication with a nitric oxide source (not shown), as previously described. Scavenged gNO exits the delivery device through a gas return outlet 532 that is in fluid communication with a gas collector (not shown), as previously described. The gas probe assembly 560 has a proximal and a distal end. The distal end is intended for insertion into the body of the patient, to allow delivery of the treatment gas (gNO) to a tumor target of a patient, and further to allow scavenging of extra or spent gNO or byproducts thereof from the vicinity of the tumor target. The proximal end is adapted for interfacing with the support body and maintaining fluid communication with the nitric oxide source of the gas collector, as previously described. The depicted delivery device 512 includes two main parts, a support body 540 and a gas probe assembly 560. The support body 540 permits the gas flow inlet 510, the gas return outlet 532 and the gas probe assembly 560 to interface with each other, providing appropriate fluid communication between the appropriate gas inflow and gas return channels.

The gas probe assembly 560, comprising an elongate shaft 562 and a proximal plastic head 564, is enclosed within the support body 540 proximally, and extends distal to the support body 540 with a shaft 562. In embodiments, the shaft 562 has a length between about 2 and about 12 inches, allowing it to be conveniently deployed to access the tumor target, for example with access through a laparoscope or other endoscope requiring a longer length (e.g., about 12 inches) and access to a more superficially located tumor target requiring a shorter length (e.g., about 2-3 inches). In certain embodiments, the shaft 562 can have a length between about 3 and about 5 inches, while in other embodiments the shaft 562 can be longer or shorter, as would be appropriate for accessing the anatomic area in which the tumor target is located. The plastic head 564 is durably affixed to the proximal end of the shaft 562, allowing the gas probe assembly 560 to be seated securely in the support body 540 when the delivery device 512 is assembled. While the plastic head 564 is shown in the depicted embodiment, in other embodiments, the head section of the gas probe assembly can be formed of any suitable material, whether metal, polymeric, or other, and can be a separate part if needed. The purpose of 564 is to attach and seal the inner channel 568 to the support body 540 and to provide a seal between the inlet fitting 542 and the support body 540.

In the depicted embodiment, shown in FIG. 5A and also shown in the cross-section taken at dotted-line X shown in FIG. 5B, the shaft 562 of the gas probe assembly 560 is formed from two concentric members: a hollow inner insufflation member 568 defining an insufflation channel 502 in its interior, and a hollow outer gas return sleeve 570 arranged coaxially surrounding the inner insufflation member 568 defining a gas return channel 504 in its interior, as previously described; in other embodiments, the arrangement of the concentric members can be reversed, with an inner gas return sleeve and an outer insufflation member, with the proximal connections arranged appropriately for gas inflow and gas evacuation. Mechanisms for controlling the rate and amount of gas inflow can be incorporated at any level of the gas inflow assembly. Advantageously, the gas probe assembly 560 is equipped with an inflow control mechanism that can be controlled by the operator, such as a valve or a stopcock that is manually operated or a digital control mechanism that responds to operator intervention. This permits the operator to directly manage the rate, velocity, and amount of gas inflow, depending on the assessment of clinical circumstances during the procedure.

At the distal end of the gas probe assembly 560 both the insufflation channel 502 and the gas return channel 504 open into a shared gas access space 506 defined by a surrounding diffusion membrane 516 (which can be similar in configuration to any of 116, 216, 316, 416a, 416b, or 416c). In embodiments, the shared gas access space 506 is flush with the end of the outer gas return sleeve 570, with the end of the inner insufflation member 568 at the same level as the end of the outer gas return sleeve 570, or with the end of the inner insufflation member 568 retracted somewhat relative to the outer gas return sleeve 570. In embodiments, the shared gas access space 506 extends out from the end of the gas probe assembly 562, for example with a gap 508 between the end of the gas probe and the distalmost portion of the diffusion membrane 516. Illustrative embodiments of the shared gas access space 506 shown in this figure are shown schematically in FIGS. 4A-D. In embodiments, the gap 508 can be between about 0.1 mm and about 10 mm in length, or between about 0.5 mm and 5 mm in length, or between about 1 mm and 2 mm in length, or variations thereof. In embodiments, the diffusion membrane 516 can be supported by ancillary supporting structures (not shown) to prevent its collapse, an arrangement that can be advantageous with different probe configurations, for example if an elongated shared gas access space 506 is desired or if a large gap 508 is desired. In embodiments, specific treatments or coatings can be applied to the membrane, for example an anticoagulant to prevent clotted blood from interfering with gas outflow or gas return.

As previously described, the shared gas access space 506 can support both the outflow of gNO towards the tumor target and the retrieval of scavenged gNO. In a preferred embodiment, the shared gas access space 506 can permit outflow of gNO towards a tumor target (not shown) and scavenging/recovery of excess gas that enters the shared access space 506 but is not to be directed to the tumor target; such outflow and scavenging/recovery can take place simultaneously or near-simultaneously, since both channels access the shared access space 506, or can take place sequentially, with scavenging following outflow in whole or in part. In this way, the outflow of gNO is balanced with scavenging, so that the dose of gas delivered to the tumor target is a therapeutically effective amount. In embodiments, the diffusion membrane 516 can have zones of differential permeability, for example so that gas delivered into the central portion of the shared gas access space 506 passes more readily through the diffusion membrane 516 to access the tumor target, while the peripheral part of the diffusion membrane 516 is less permeable, trapping the excess gas that spills out towards the peripheral portion of the shared access space 506 to facilitate its removal. In an embodiment, the diffusion membrane 516 can be affixed to the distal end of the outer gas return sleeve 570, using affixation methods familiar in the art. For example, the diffusion membrane can be glued onto a metal external surface of the outer gas return sleeve 570. If the outer gas return sleeve 570 has a polymeric overcoating, the diffusion membrane can be melted into the overcoating, or both layers can be glued to each other. Other affixation methods will be apparent to skilled artisans using no more than routine experimentation.

The embodiment depicted in FIG. 5A shows a distal tip structure integrated with the gas probe assembly 562, comprising a shared gas access space 506, a diffusion membrane 516 and a gap 508 between the end of the gas probe and the distalmost portion of the diffusion membrane 516. In other embodiments, a distal tip structure including the shared gas access space 506, the diffusion membrane 516, and the gap 508 can be fabricated as a separate attachable component that is removably affixable to the gas probe assembly 562. In embodiments, the separate distal tip structure can be equipped with sharpened edges that facilitate penetration into selected tissues such as solid tumors or cyst walls. In embodiments, the separate distal tip structure can be screwed on or attached via a snap-on mechanism or any other removable affixation known in the art. The separate distal tip structure can be fabricated as a single-use component, which can be included in a kit of components for use in an individual patient. The separate distal tip structure can be available in a range of sizes to fit the needs of the patient and/or to conform to the size of the lesion being treated. The shared gas access space 506, the diffusion membrane 516, and the gap 508 can all be configured individually in different distal tip structures, with sizes and shapes to match the particular treatment needs for the specific patient.

The gas probe assembly structure 560 is insertable into the support body, which permits its connection to the conduits for gas inflow 510 and gas return 532. The support body 540 is formed as a solid unit penetrated by a central hollow passage along its long axis and further provided with two screw sockets that receive the gas flow inlet assembly 542 and the gas return outlet assembly 544. Preferably the solid support body 540 and the assemblies it receives are fabricated from metal, although polymeric versions of any of the components disclosed herein can be engineered in keeping with the principles of the invention. Each of the gas flow inlet assembly 542 and gas return outlet assembly 544 supports a cylindrical hollow passage along its long axis (a gas inflow passage 514 and a gas return passage 534, respectively) and each has a screw connection that interfaces with the screw threads in its respective socket on the support body 540.

To assemble the delivery device 512, the gas probe assembly 560 is passed from proximal to distal through the support body 540. The central hollow passage of the support body 540 is dimensionally adapted to permit the passage of the distal portion of gas probe assembly 560 therethrough. The proximal head 564 engages with the base of the gas inflow socket when the gas probe assembly 560 is properly positioned within the support body 540. Following the positioning of the gas probe assembly 560, the gas inflow assembly 542 and the gas return outlet assembly 544 can be connected. Each of these assemblies can be formed with a screw connector to interface with the appropriate socket on the support body 540. At the base of each socket is a compressible O-ring, so that the process of screwing in the assembly compresses the O-ring to form a gas-tight seal between the connected components. As the gas inflow assembly 542 is screwed into its socket in the support body 540, the plastic head 564 of the gas probe assembly 560 is compressed, further reinforcing the gas-tight seal between these two components. The distal end of the support body 540 has a male screw configuration that interfaces with a female screw socket on a cylindrical sealing fastener 548, where the base of the socket on the cylindrical sealing fastener 548 supports a compressible O-ring 546. Tightening the screw connection between the distal end of the support body 540 and the cylindrical sealing fastener 548 compresses the O-ring 546 and creates a seal between the fastener 548 and the contact surface on the outer channel of the gas probe assembly 560 to prevent passage of moisture or fluids, including gas; this procedure also attaches and seals the outer channel of the probe assembly 560 to the support body 540. While a screw connection is advantageous for producing a desirable gas-tight seal, other connections of the cylindrical sealing fastener 548 and the support body 540 can be envisioned, such as a press fit or other fitting methods familiar to skilled artisans.

The gas flow inlet assembly 542 includes a gas flow inlet 510 and an interior gas inflow passage 514 that are in fluid communication with the insufflation channel 502 within the gas probe assembly 560 when the delivery device 512 is assembled. Similarly, the gas return outlet assembly 544 includes a gas return outlet 532 and an interior gas return passage 534 that are in fluid communication with the gas return channel 504 in the gas probe assembly 560. A Luer lock coupling is provided on the gas flow inlet 510 to couple the delivery device 512 to the inflow side of the overall nitric oxide treatment system (as shown in previous Figures). A coupling on the gas return outlet 532 couples the delivery device 512 to the outflow side of the overall nitric oxide treatment system (as shown in previous Figures). A Luer lock coupling is optional for the gas return outlet, since the outflow side is not subject to high pressures and flows similar to those on the inflow side, and the outflow side can operate at low gas pressures or negative gas pressures, thus entailing less risk of gas extravasation; a variety of couplings for the gas return outlet can be envisioned by skilled artisans, consistent with the principles of these systems and methods. Having a different connection on the outlet fitting as compared to the inlet fitting also minimizes the possibility of accidentally reversing the inlet and out connections.

Once assembled and connected to the source of gNO and connected to the gas collector (as depicted schematically in FIG. 1) the delivery device 512 permits gNO to be directed into the gas inflow inlet 510 and into the gas inflow passage 514 to enter the insufflation channel 502 in the elongate shaft 562 of gas probe assembly 560 and to proceed distally to reach the tumor target (not shown). In the depicted embodiment, excess or spent gNO and any byproducts thereof are evacuated into the gas return channel 504 in the elongate shaft 562 of the gas probe assembly 560 and return proximally to enter the gas return passage 534 in the gas return outlet assembly 544, passing through the gas return outlet 532 and thereby entering the gas collector. Optional mechanisms for controlling the rate and amount of gas return can be incorporated at any level of the gas return assembly, allowing restriction of outflow of spent gas so that an appropriate amount of gNO remains in contact with the area being treated; such mechanisms can be mechanically controlled, such as a valve or stopcock on the outflow end of the system that is manually operated, or they can be integrated within the overall gas return assembly and controlled digitally or automatically without manual intervention.

While intended for use in conjunction and connected to with a gNO treatment system, the delivery device 512, comprising the support body 540 and the gas probe assembly 560 can be presented for clinical use in a disassembled state, and can be offered as a kit comprising some or all of its disassembled components. The separation of the delivery device 512 into its separate components facilitates the process of sterilizing these components for use and reuse. The support body 540 and its attachable screw-in connectors (the gas inflow inlet assembly 542, the gas return outlet assembly 544 and the sealing fastener 546) are separate units that can be presented individually in a sterile condition, for example in a kit, to be assembled by a clinician on the sterile field. Similarly, the gas probe assembly 560 is a separate structure, also available in a kit alone or in combination with the support body components. The gas return assembly can include a biofilter (not show) to prevent particulate matter (cells, tissues, debris, etc.) from entering the gas collector, or other filters as appropriate. Advantageously, such filters can be disposable and can be included as part of a kit of single use components to be employed with an individual patient. In embodiments, the biofilter can be positioned in any convenient location to restrict the ingress of particulate matter into the gas collector, for example, at an entry point for the scavenged gas, or within the gas return channel 504, or within the gas return passage 534.

The gas probe assembly 560 can be inserted into the support body 540 at the point of use to form the assembled delivery device 512. In embodiments, the gas probe assembly 560 itself can be separated into separate components, with the inner hollow cylinder (the insufflation member 568 as depicted) being insertable into and affixable to the outer hollow cylinder (the outer gas return sleeve 570 as depicted). In embodiments, the gas probe assembly is equipped with a flared head 564 surrounding at least a portion of its proximal end (here the insufflation member 568) that facilitates its secure seating in the socket for the gas flow inlet assembly 542, as described in more detail below. The flared head 564 can be formed of metal or can be formed from a compressible material, thereby allowing for more secure seating in the socket as the gas flow inlet assembly 542 is screwed into place. The gas probe assembly 560 can be prepared for use by inserting its component inner cylinder into the outer cylinder at the point of use, following which the assembled gas probe assembly 560 can be inserted into the support body 540 with subsequent attachment of the screw-in connectors (the gas inflow inlet assembly 542, the gas return outlet assembly 544 and the sealing fastener 546) into the support body 540.

One or more of the individual components of the delivery device 512 can be disposable. For example, the gas probe assembly 560 can be fabricated from materials that render portions of this component disposable or that allow the entire gas probe assembly 560 to be disposable. In embodiments, some or all of the components of the delivery device 512 are made from metal in whole or in part to facilitate reuse and resterilization before subsequent use. Resterilization of the reusable components can be accomplished using techniques familiar in the art, first by disassembling the delivery device 512 after its use. Certain components that are susceptible to wear after a single use, such as O-rings, can be provided as single-use-only accessories, with a fresh supply of these accessories being inserted into their predetermined locations each time prior to assembling the support body and its connectors. For example, appropriately sized O-rings can be positioned in the base of the receiving sockets prior to screwing the gas inflow inlet assembly 542, the gas return outlet assembly 544 and the sealing fastener 546 into their predesignated positions relative to the support body 540.

In embodiments, the gas probe assembly 560 is a rigid metal structure with two metal channels, as described herein in greater detail, which can be arranged coaxially as depicted herein, or can be arranged eccentrically, in parallel, or in any other suitable geometric relationship. In embodiments, the insufflation and gas return members within the gas probe assembly 560 can be made of rigid or flexible polymeric materials, provided that such materials are impervious to the passage of the gNO and its gaseous byproducts. Use of polymeric materials can permit the gas probe assembly 560 or its components to be fabricated for single-use, thus avoiding the difficulties of cleaning and sterilizing the small-bore passages of the shaft 562 for reuse. In embodiments, one or more of the components can be presented for single use in a kit. In embodiments, the gas probe assembly 560 can be fabricated in whole or in part from flexible polymeric materials impervious to gNO and its gaseous byproducts, with the flexibility of the materials permitting the insertion of the device through a catheter or other flexible tube or scope, in order to reach hard-to-access anatomic areas. For example, a flexible gas probe assembly 560 can be used to navigate tortuous passages such as the nasopharyngeal sinuses, or the epidural or subdural spaces. In embodiments, a flexible gas probe assembly 560 can be steerable using optional steering mechanisms. In embodiments, the flexible gas probe assembly 560 can be equipped with radiopaque trackers that allow its progress and passage to be monitored fluoroscopically. A flexible gas probe assembly 560 can also be adapted for intravascular use as part of a catheter-based insertion system, recognizing that a treatment area accessed by an intravascular approach requires additional segregation to avoid intravascular dissemination of the gNO treatment agent.

FIG. 5C provides a close-up view of a portion of the embodiment depicted in FIG. 5A contained within the dotted-line “Y”. The close-up view in FIG. 5C is provided in order to illustrate more clearly the relationship of the concentric members forming the elongate shaft 562b of the gas probe assembly as they are arranged within the support body 540b.

The close-up view of FIG. 5C (numbered similarly to FIGS. 5A and 5B) shows the shaft 562b of the gas probe assembly formed from two concentric members: a hollow inner insufflation member 568b defining an insufflation channel 502b in its interior, and a hollow outer gas return sleeve 570b arranged coaxially surrounding the inner insufflation member 568b defining a gas return channel 504b in its interior, as previously described. Also as previously described, the gas probe assembly structure is insertable into the support body 540b, which permits its connection to the conduits for gas inflow 514b and for gas return 532b.

As shown in this figure in more detail, the gNO enters the depicted delivery device (512, as shown in FIG. 5A) through the gas inflow passage 514b, passing through the gas inflow assembly 542b to enter the insufflation channel 502b within the insufflation member 568b. Also as shown in this figure in more detail, excess or spent gNO and any byproducts thereof are evacuated into the gas return channel 504b enclosed within the gas return sleeve 570b and return proximally to enter the gas return passage 534b in the gas return outlet assembly 544b, passing through the gas return outlet 532b and thereby entering the gas collector. As shown in this figure in more detail, the gas return channel 504b is in fluid communication with the gas return passage 534b, while the gNO on the inflow side is entirely contained within the gas inflow passage 514b and the insufflation member 568b. In the depicted embodiment, a gap 580b in the gas return sleeve 570b allows the gas in the gas return channel 504b to enter the gas return passage 534b for subsequent collection.

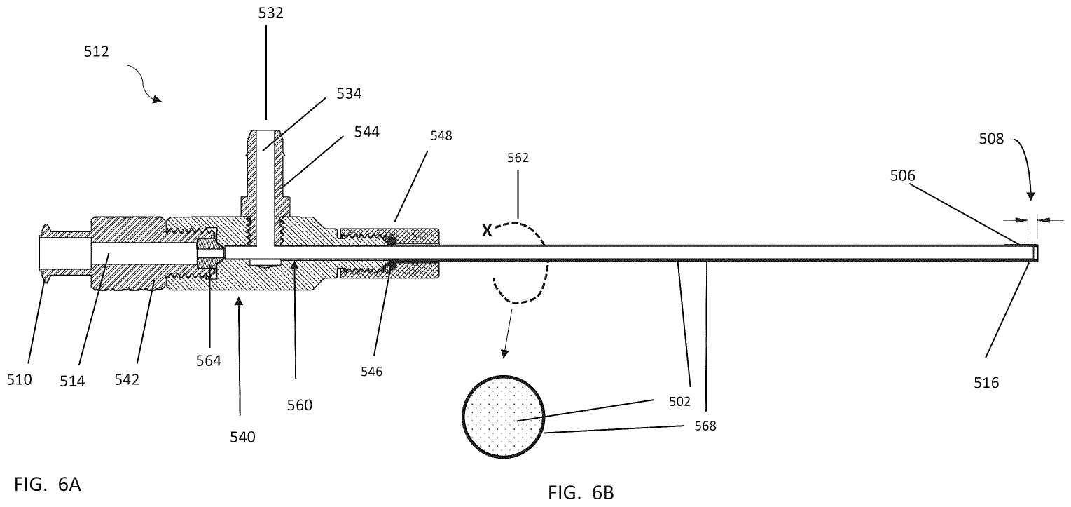

FIGS. 6A and 6B depict a further embodiment of the gas probe assembly 560 which does not include the gas return sleeve 570 arranged coaxially surrounding the insufflation member 568. Accordingly, as depicted in FIG. 6A, the gas flow inlet assembly 542 is now in fluid communication with the gas return outlet assembly 544. In this regard, once assembled and connected to the source of gNO and connected to the gas collector (as depicted schematically in FIG. 1), the delivery device 512 permits gNO to be directed into the gas inflow inlet 510 and into the gas inflow passage 514 to enter the channel 502 in the elongate shaft 562 of gas probe assembly 560 and to proceed distally to reach the tumor target via the diffusion membrane 516, with any excess gNO or byproducts thereof being evacuated into the gas return passage 534 in the gas return outlet assembly 544. Specifically, the gNO is provided to the tumor target via diffusion of the gNO from a high concentration area near the gas flow inlet assembly 542 to a lower concentration area near the distal end of the gas probe assembly 560, and then through the diffusion membrane 516. In embodiments, the diffusion membrane 516 is configured such that gNO is diffused through the diffusion membrane 516 solely in the direction of the tumor target. The gas that does not diffuse through the diffusion membrane 516 (e.g., the excess gas) and remains within the channel 502 in the elongate shaft 562 is subsequently evacuated from the channel 502 through the gas return passage 534 in the gas return outlet assembly 544.

The embodiment depicted in FIGS. 6A and 6B can result in smaller-diameter probe assemblies 560, which can be used to treat smaller-sized tumors.

### Section 3: Exemplary Methods of Use

The delivery device as disclosed herein is suitable for use in conjunction with a system for treating a tumor mass, wherein a therapeutic amount of gNO is delivered to a tumor target, and the excess gNO and its gaseous byproducts are scavenged from the delivery area. As would be appreciated by skilled artisans, the delivery device can be inserted into the body of a patient in need of gNO treatment through any modality of surgical, endoscopic, robotic, or catheter-directed access. In embodiments, the device provides a rigid shaft suitable for advancement or positioning to reach the tumor target, while in other embodiments, the shaft can be formed as a flexible unit for advancement using catheters or other flexible delivery modalities. In embodiments, the shaft can be formed as a rigid structure adapted for penetrating the tumor mass to deliver the gNO internally to the tumor mass. The distal end of the shaft can be designed to facilitate such penetration, for example by being formed as a rounded, blunt or sharpened end as previously described. In other embodiments, the delivery device can include a retractable outermost sleeve with a sharpened distal edge that can pierce the tumor mass and then be displaced proximally to uncover the distal ends of the insufflation channel and the return sleeve, both covered by the diffusion membrane as described herein. In yet other embodiments, the tip of the retractable outermost sleeve can be equipped with rounded instead of sharpened edges, so that it can be used to dissect bluntly the tissues surrounding the tumor mass or the tumor target itself, rather than incising them.

In embodiments, the elongate shaft permits simultaneous, near-simultaneous, or sequential gas delivery and scavenging of excess gas before such excess gas can access the tissues surrounding the delivery device. In embodiments, the distal end of the elongate shaft is adapted for positioning directly against the tumor, thereby forming a seal, or is adapted for penetrating the tumor, for example with a blunt or sharpened tip that is insertable into the tumor with the gNO being delivered into the tumor through openings in the side of the elongate shaft. In an embodiment, the elongate shaft can contact the tumor directly and administer gNO thereto and then, after the gNO is administered, any gNO remaining in the delivery device, for example, the elongate shaft is scavenged.

In embodiments, the insufflation channel and return sleeve share a gas access channel, permitting simultaneous, near-simultaneous, or sequential gas delivery and scavenging of excess gas before such excess gas can access the tissues surrounding the delivery device. In embodiments, one or more of the distal ends of the insufflation channel and the return sleeve are adapted for positioning directly against the tumor, thereby forming a seal, or are adapted for penetrating the tumor, for example with a blunt or sharpened tip that is insertable into the tumor with the gNO being delivered into the tumor through openings in the side of the insufflation channel. In an embodiment, the insufflation channel can contact the tumor directly and administer gNO thereto, with the return sleeve being available to scavenge any extra or spent gNO, and remove any byproducts thereof from the region surrounding the tumor mass.

In embodiments, the size of the gas probe assembly is selected to conform to the size of the tumor mass being treated. While small diameters are advantageous for the inner and outer channels of the gas probe assembly (e.g., with each channel having an inner diameter between about 0.5 and 10 mm depending on the dimensions of the lesion being treated, machined to permit the inner cylinder to be inserted into the outer cylinder during probe assembly while providing an adequate channel in the outer cylinder to allow its intended use for gas flow), a larger diameter probe is also consistent with the principles of the invention, as can be envisioned by artisans of ordinary skill in the field.

A smaller diameter shaft allows insertion into small anatomic spaces, and inherently restricts the flow of the gNO to the target area, thus reducing the risk of gNO exposure to normal tissues. A smaller diameter shaft can also afford more flexibility in use with conventional endoscopic instrumentation. However, the smaller diameter shaft may not be adequate for treating a larger tumor mass, and/or may require numerous repositionings. Therefore, it is understood that a gas probe assembly can be selected having a shaft diameter that is adequate to treat the specific tumor using the methods of the invention. Advantageously, the diameter of the shaft is selected to treat at least a region of the tumor without delivering gNO to normal tissues.

In embodiments, the diameter of the shaft channels is smaller, or is substantially smaller, than the size of the tumor; in other embodiments, the diameter of the shaft channels is selected to fit the size and/or shape of the tumor. In embodiments, the inner insufflation member has a diameter that is smaller or is substantially smaller, than the outer return sleeve diameter, thus targeting the tumor more directly while permitting a wider uptake space for the scavenging of excess or spent gNO, or byproducts thereof.

The delivery device as disclosed herein is intended for use in conjunction with a system for treating a tumor mass, wherein a therapeutic amount of gNO is delivered to a tumor target and the excess gNO and its gaseous byproducts are scavenged from the delivery area. The delivery device therefore has an insufflation function (delivering the gNO) and a scavenging function (removing excess, spent, or residual gNO or their byproducts or its carrier gas i.e. nitrogen), both as described above in conjunction with exemplary embodiments. Methods of locally treating a tumor mass with gNO can include the steps of providing a treatment system for generating gNO and contacting the tumor mass or portion thereof with the gNO, wherein the step of contacting the tumor mass takes place by delivering the gNO through the delivery device as disclosed herein to reach the tumor target. The methods of treatment further include the removal of extra, residual, or spent gNO from the region of the tumor target along with any gaseous byproducts or carrier gas of such treatment, which can include the creation of a lower pressure environment within the gas return channel of the delivery device to cause, facilitate, or expedite such scavenging. Advantageously, local treatment of the tumor target triggers an abscopal effect that treats tumor masses distant from the tumor target.

The delivery device can be used to administer gNO locally to a tumor target in a variety of ways. In an embodiment, the local administration of gNO to the tumor target using the delivery device can be accomplished by (1) contacting the tumor target with the distal end of the delivery device or by bringing the distal end of the delivery device into therapeutic proximity to the tumor target, (2) passing the gNO from a nitric oxide source through the delivery device so that the gNO contacts the tumor target, and (3) simultaneously or sequentially scavenging spent or excess gNO and byproducts from the region surrounding the tumor target. The step of contacting the tumor target can further include penetrating the tumor target and embedding the distal end of the delivery device in the tumor target. The step of scavenging can comprise removing spent gNO and byproducts from the region surrounding the tumor target or from within the tumor target itself. The step of scavenging can also comprise removing excess gNO from the distal end of the delivery device before such excess gNO passes into the tumor target or the region surrounding the tumor target. Local administration of the gNO can be performed at a constant rate, at a variable rate, and/or pulsed intermittently over a preselected time period; local administration can be accomplished in a single session or on multiple occasions as part of an overall treatment plan for locoregional and abscopal distal control of the tumor. Local administration can also be performed with different concentrations of gNO at different time intervals, or with different durations of exposure and rest periods between the pulses. During the rest period, the extra, residual, or spent gNO or their byproducts can be scavenged from the site of administration using the scavenging function of the delivery device.

As used herein, the term “local administration” refers to the act of providing the gNO to the tumor target so that at least some of the gNO provided contacts the tumor cells. Without being bound by theory, it is envisioned that at least some of the gNO applied in therapeutic proximity enters the tumor cells by diffusion, exerting a therapeutic effect on such tumor cells. Local administration can take place by applying the gNO directly to a surface of the tumor mass, or inserting the gNO into the tumor mass, or insufflating the gNO into a region otherwise in therapeutic proximity to the tumor target (whether delivered internally into the tumor or whether delivered to the tumor surface or the region surrounding the tumor), so that the gNO can exert a therapeutic effect on the tumor mass. Such therapeutic proximity is determined by the effect on the tumor of a given volume, flow, and concentration the delivered gNO; as examples, therapeutic proximity can involve delivery of gNO within up to 0.5 cm, 1.0 cm, 1.5 cm, or 2.0 cm in relation to at least one of the surfaces of the tumor mass. In other examples, therapeutic proximity involves the delivery of gNO to the surface of the tumor mass or to the interior of the tumor mass.

In embodiments, the local administration to the tumor mass comprises a balancing between the amount of gNO outflow from the insufflation channel versus the amount of gNO uptake by the return channel, wherein the uptake of gNO removes excess and spent gNO from the delivery area, allowing the desired amount to cross a boundary membrane such as a selectively permeable membrane to provide a therapeutically effective dose of gNO to the tumor mass without overexposure of the tumor mass to the gNO and while minimizing the contact of the gNO with normal tissues in or near the treatment region.

As mentioned above, the amount of gNO administered affects the efficacy of local administration. In embodiments, a high dose of gNO is administered, determined by the mass of gNO that is locally delivered to the tumor mass in relation to the mass of the tumor, which is based on the concentration of gNO in the gas flow directed towards the tumor, the flow rate of the gas flow, and the duration of the gas flow. In embodiments, the gNO gas flowrate should be at a rate to make up for the flowrate of gNO leaving through the diffusion membrane and returning through the gas return channel in order to maintain a desired concentration of gNO at the membrane, thereby maintaining (preventing a drop in) the diffusion rate of gNO across the membrane.

The gNO mass delivered to the tumor is therefore a function of the gNO concentration (ppm), volumetric flow rate (ml/min) and time duration of delivery; the gNO mass can be calculated using the known ideal gas equation, PV=nRT, wherein P is the pressure, V is the volume, n is the number of moles, R is the gas constant and T is the temperature. While the amount of gNO delivered is determined clinically based on the therapeutic needs of the individual patient, in embodiments, the following exemplary amounts of local administration can be selected: gNO can be locally administered in an amount of 0.1 to about 300 mg (including any intermediate values and subranges therebetween) per 1 cm³ of tumor pass, per administration, recognizing the potential for the administered gNO to have an adverse effect on the healthy tumor tissues in the vicinity of the tumor mass. The scavenging function of the delivery device can be adjusted to remove the extra, residual, or spent gNO or their byproducts so that a larger immediate high dose of gNO can be administered to the tumor mass while minimizing its impact on surrounding healthy tissues in the treatment region.

The method of using the inventive delivery device for treatment is contingent on the overall gNO delivery system, as described above. The method of using the inventive delivery device is also contingent upon the surgical or other method selected for accessing the tumor target. In certain embodiments, the tumor target is accessed by direct visualization techniques such as an open surgical approach or a limited surgical access or keyhole approach. In such cases, no specialized insertion apparatus is required, as the delivery device can itself be handheld or otherwise retained by the surgeon in contact with the tumor target or in therapeutic proximity thereto, and/or can be embedded directly into the tumor by the surgeon and retained therein manually or by optional equipment. Optional equipment to support the delivery device within the body at the appropriate distance from or within the tumor target can also be employed in addition to or instead of manual support by the operator. If less invasive methods are employed to visualize and access the tumor, the delivery device can be employed in conjunction with appropriate visualization and manipulation equipment, whether operated manually, robotically, or some combination thereof. For example, the delivery device can be used in conjunction with insertion equipment familiar to practitioners for introducing a probe into a treatment area through minimally invasive or endoscopic means.

Needle localization techniques, fluoroscopic guidance, and other visualization and manipulation techniques known in the art are consistent with the systems and methods disclosed herein. In an exemplary embodiment, the tumor target can be identified radiologically or fluoroscopically, with placement of a localizing needle therein. The needle can serve as a guide for a cannula, optionally equipped with a trocar, to be inserted into the tumor mass. The cannula can then guide the insertion of the delivery device into the tumor target. Such access and subsequent treatment can be employed for solid tumors embedded in surrounding healthy tissue, for example, for a breast tumor. In another exemplary embodiment, for tumor targets within a body cavity or a body passage endoscopic access to the tumor can be provided. Using techniques familiar in the art, the endoscope can be used to identify and localize the tumor. The endoscope can contain a channel permitting the insertion of the delivery device through the endoscope while optionally permitting visualization of the tumor target. Alternatively, a first endoscope can permit visualization of the tumor target and surrounding tissues, while a separate, second endoscope can provide the introduction channel that enters the body cavity or body passage to allow positioning of the delivery device in therapeutic proximity to the tumor mass. The first endoscope can provide direct visualization or remote imaging of the tumor target and can provide feedback and information about the position of the delivery device to optimize the accurate delivery of the gNO and the scavenging of the gNO and its byproducts. Optionally, the endoscopic system described above can be combined with conventional techniques for minimally invasive surgical procedures, allowing for other ancillary and routine aspects of minimally invasive surgery to be performed, such as electrocautery, irrigation and evacuation, collection of biopsy samples, introduction of other therapeutic agents, and the like.

Endoscopic systems designed for flexible use in tortuous channels can be combined with flexible versions of the delivery device; such systems can be useful in endoscopic approaches such as flexible bronchoscopy, colonoscopy, and the like, where the endoscope can be passed through a body lumen to access the tumor target and the delivery device can be directed through the endoscope to treat the tumor target with the gNO and carry out its scavenging function. In such a situation, the tumor target can be protruding into the body lumen without an enveloping or encasing rim of healthy tissue. In certain embodiments, the delivery of gNO into such a region can readily result in extravasation from the delivery site. Under these circumstances, the scavenging function can be adjusted to maximize the retrieval, removal, or recovery of gNO and its byproducts from the site or to minimize such extravasation, for example by implementing or increasing the low-pressure environment within the gas return channel, e.g., introducing a partial vacuum therein, although any pressure lower than atmospheric pressure can be considered a low-pressure environment for the purposes of these systems and methods. Under certain circumstances, auxiliary structures on the endoscopic mechanism can be employed, for example a retractable hood that partially or wholly surrounds the tumor target, thereby confining the diffusion of gNO and its byproducts to a discrete region around the tumor target and facilitating the scavenging process. Both the endoscope and the delivery device can be directable and positionable by the operator via controls positioned more proximally on the device, as would be familiar to skilled artisans.

While numerous examples have been provided above, and exemplary embodiments of delivery devices consistent with these systems and methods have been described, it is understood that the delivery device is consistent with a variety of gNO treatment systems, effecting insufflation and scavenging of gNO. Modification of the delivery device disclosed herein to conform to the requirements of specific gNO treatment systems are embraced by the present invention. It is further understood that the delivery device and the gNO treatment systems that it supports are consistent with a wide range of interventional treatment modalities for tumors, including open surgeries, less invasive surgeries, minimally invasive procedures, endoscopic procedures, catheter-directed procedures, and other interventional techniques that are devised in the future, whether directly or indirectly human-mediated, or mediated by remote human interaction, or performed robotically, by computer direction, or otherwise. It will be apparent to skilled artisans that numerous modifications, variations, and alternatives can be devised for the delivery system, the gNO treatment system, and the techniques for accessing the tumor target, all falling within the spirit and broad scope of the appended claims.

1. A medical gas delivery device for delivering a medical gas to a tumor mass while minimizing exposure to spent gas or excess gas, the device comprising a support body coupled with an elongate shaft, wherein the elongate shaft comprises a distal tip structure at its distal end, and wherein the distal tip structure comprises a distal end of the elongate shaft; and
wherein a selectively permeable diffusion membrane encloses the distal end of the elongate shaft.

2. The device of claim 1, wherein the elongate shaft comprises at least 2 channels, wherein at least one channel is an insufflation channel that passes from proximal to distal within the elongate shaft and at least one channel is a gas return channel that passes from distal to proximal within the elongate shaft.

3. The device of claim 2, wherein the distal tip structure comprises the distal end of the insufflation channel and a distal end of the gas return channel.

4. The device of claim 2, wherein the selectively permeable diffusion membrane encloses the distal end of the insufflation channel and the distal end of the gas return channel.

5. (canceled)

6. (canceled)

7. The device of claim 1, wherein the distal tip structure further comprises one or more gas access spaces in fluid communication with the distal end of the insufflation channel.

8. The device of claim 2, wherein the distal tip structure further comprises one or more gas access spaces in fluid communication with the distal end of the insufflation channel and the distal end of the gas return channel.

9. The device of claim 1, wherein the distal end of the insufflation channel contains one or more openings on its side for gas delivery.

10. (canceled)

11. The device of claim 7, wherein the one or more gas access spaces form a single shared gas access space, and the single shared gas access space is in fluid communication with the insufflation channel and the gas return channel.

12. (canceled)

13. (canceled)

14. The device of claim 1, wherein the insufflation channel is disposed within the gas return channel.

15. (canceled)

16. The device of claim 1, wherein:
(a) the support body comprises an inflow socket adapted for connection with a gas flow inlet assembly, wherein the gas flow inlet assembly defines a gas flow inlet passage within, and wherein the gas flow inlet passage establishes fluid communication between a source of medical gas and the insufflation channel; and
(b) the support body comprises an outflow socket adapted for connection with a gas return outlet assembly, wherein the gas return outlet assembly defines a gas return outlet passage.

17. The device of claim 16, wherein the gas flow inlet assembly is connectable proximally to the source of medical gas and is connectable distally to the inflow socket.

18. The device of claim 16, wherein the gas return outlet assembly is connectable proximally to the gas collector and is connectable distally to the outflow socket.

19-22. (canceled)

23. The device of claim 1, wherein the support body encloses the proximal end of the elongate shaft.

24. The device of claim 23, wherein the proximal end of the elongate shaft comprises a flared head dimensionally adapted for seating in a distal end of the inflow socket.

25. The device of claim 24, wherein the flared head is formed from a compressible material.

26-31. (canceled)

32. The device of claim 1, wherein the elongate shaft is adapted for positioning through an endoscope to access a treatment region.

33. The device of claim 8, wherein the one or more gas access spaces comprise an insufflation gas access space in fluid communication with the insufflation channel and a return gas access space in fluid communication with the gas return channel, and wherein the insufflation gas space and the return gas access space are not in fluid communication with each other.

34. The device of claim 1, wherein the medical gas is therapeutic gaseous nitric oxide (gNO).

35. A system for delivering therapeutic gNO to a tumor mass while minimizing exposure to spent gas or excess gas, comprising a source of therapeutic gNO, and a medical gas delivery device according to claim 1.

36-44. (canceled)

45. A kit for use with the system of claim 35, comprising:
(a) a support body of a gas delivery device and
(b) an elongate shaft of a gas delivery device insertable into the support body to form the gas delivery device,
wherein each of the support body and the elongate shaft is provided in the kit as a separate component.

46-71. (canceled)
